SEQUENCE LISTING (21 sequences)

SEQ ID NO: 1 - protein, 8 aa, artificial sequence
  4-1BB Heavy Chain CDR 1
GYTFTSYW

SEQ ID NO: 2 - protein, 8 aa, artificial sequence
  4-1BB Heavy Chain CDR 2
IYPGSSTT

SEQ ID NO: 3 - protein, 14 aa, artificial sequence
  4-1BB Heavy Chain CDR 3
ASFSDGYYAYAMDY

SEQ ID NO: 4 - protein, 6 aa, artificial sequence
  4-1BB Light Chain CDR  1
QDISNY

SEQ ID NO: 5 - protein, 3 aa, artificial sequence
  4-1BB Light Chain CDR  2
YTS

SEQ ID NO: 6 - protein, 9 aa, artificial sequence
  4-1BB Light Chain CDR  3
QQGYTLPYT

SEQ ID NO: 7 - protein, 8 aa, artificial sequence
  OX40 Heavy Chain CDR1
GFTLSYYG

SEQ ID NO: 8 - protein, 8 aa, artificial sequence
  OX40 Heavy Chain CDR2
ISHDGSDK

SEQ ID NO: 9 - protein, 7 aa, artificial sequence
  OX40 Heavy Chain CDR3
SNDQFDP

SEQ ID NO: 10 - protein, 6 aa, artificial sequence
  OX40 Light Chain CDR1
NIGSKS

SEQ ID NO: 11 - protein, 3 aa, artificial sequence
  OX40 Light Chain CDR2
DDS

SEQ ID NO: 12 - protein, 11 aa, artificial sequence
  OX40 Light Chain CDR3
QVWDSSSDHVV

SEQ ID NO: 13 - protein, 727 aa, artificial sequence
  4-1BB x OX40 bispecific antibody
EVQLVQSGAEVKKPGASVKVSCKASGYTFTSYWMNWVRQAPGQGLEWMGNIYPSGGSTNYAQKFQGRVTMTVDTSTSTVYMELSSLRSEDTAVYYCASFSDGYYAYAMDYWGQGTLVTVSSGGGGSGGGGSGGGGSGGGGSEIVMTQSPATLSLSPGERATLSCRASQSVSSYLNWYQQKPGQAPRLLIYYASRRHTGIPARFSGSGSGTDFTLTISSLQPEDFAVYYCQQGYNLPYTFGQGTKVEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSSYVLTQPPSVSVAPGKTARITCGGNNIGSKSVNWFQQKPGQAPVLVVYDDSGRPSGIPERFSGSTSGNTATLTISRVEAGDEADYYCQVWDSSSDHVVFGGGTKLTVLGGGGSGGGGSGGGGSGGGGSQVQLVESGGGVVQPGRSLRLSCAASGFTLSYYGMHWVRQAPGKGLEWVAAISHDGSDKYYADSVKGRFTISRDNSKNRLYLQMNSLRAEDTAVYYCSNDQFDPWGQGTLVTVSSR

SEQ ID NO: 14 - protein, 121 aa, artificial sequence
  4-1BB Variable Heavy Chain  Amino Acid Sequence
EVQLVQSGAEVKKPGASVKVSCKASGYTFTSYWMNWVRQAPGQGLEWMGNIYPSGGSTNYAQKFQGRVTMTVDTSTSTVYMELSSLRSEDTAVYYCASFSDGYYAYAMDYWGQGTLVTVSS

SEQ ID NO: 15 - protein, 107 aa, artificial sequence
  4-1BB Variable Light Chain
EIVMTQSPATLSLSPGERATLSCRASQSVSSYLNWYQQKPGQAPRLLIYYASRRHTGIPARFSGSGSGTDFTLTISSLQPEDFAVYYCQQGYNLPYTFGQGTKVEIK

SEQ ID NO: 16 - protein, 248 aa, artificial sequence
  scFv
EVQLVQSGAEVKKPGASVKVSCKASGYTFTSYWMNWVRQAPGQGLEWMGNIYPSGGSTNYAQKFQGRVTMTVDTSTSTVYMELSSLRSEDTAVYYCASFSDGYYAYAMDYWGQGTLVTVSSGGGGSGGGGSGGGGSGGGGSEIVMTQSPATLSLSPGERATLSCRASQSVSSYLNWYQQKPGQAPRLLIYYASRRHTGIPARFSGSGSGTDFTLTISSLQPEDFAVYYCQQGYNLPYTFGQGTKVEIK

SEQ ID NO: 17 - protein, 114 aa, artificial sequence
  OX40 Variable Heavy Chain
QVQLVESGGGVVQPGRSLRLSCAASGFTLSYYGMHWVRQAPGKGLEWVAAISHDGSDKYYADSVKGRFTISRDNSKNRLYLQMNSLRAEDTAVYYCSNDQFDPWGQGTLVTVSS

SEQ ID NO: 18 - protein, 108 aa, artificial sequence
  Variable Light Chain
SYVLTQPPSVSVAPGKTARITCGGNNIGSKSVNWFQQKPGQAPVLVVYDDSGRPSGIPERFSGSTSGNTATLTISRVEAGDEADYYCQVWDSSSDHVVFGGGTKLTVL

SEQ ID NO: 19 - protein, 243 aa, artificial sequence
  OX40 scFv
SYVLTQPPSVSVAPGKTARITCGGNNIGSKSVNWFQQKPGQAPVLVVYDDSGRPSGIPERFSGSTSGNTATLTISRVEAGDEADYYCQVWDSSSDHVVFGGGTKLTVLGGGGSGGGGSGGGGSGGGGSQVQLVESGGGVVQPGRSLRLSCAASGFTLSYYGMHWVRQAPGKGLEWVAAISHDGSDKYYADSVKGRFTISRDNSKNRLYLQMNSLRAEDTAVYYCSNDQFDPWGQGTLVTVSSR

SEQ ID NO: 20 - protein, 15 aa, Artificial Sequence
  Linker
GGGGSGGGGSGGGGS

SEQ ID NO: 21 - protein, 5 aa, artificial sequence
  Linker
  misc_feature (1)..(5): Wherein the sequence can be repeated between 1 to 5 times
GGGGS

(19) United States
(12) Patent Application Publication
(10) Pub. No.: US 2024/0150483 A1
(43) Pub. Date: May 9, 2024

(54) COMPOSITIONS COMPRISING 4-1BB and OX40 BINDING PROTEINS AND METHODS OF USE

(71) Applicant: Aptevo Research and Development LLC, Seattle, WA (US)

(72) Inventor: David Leonard BIENVENUE, Seattle, WA (US)

(21) Appl. No.: 18/546,751

(22) PCT Filed: Feb. 17, 2022
(86) PCT No.: PCT/US2022/016776

§ 371 Date: Aug. 16, 2023

Related U.S. Application Data
(60) Provisional application No. 63/150,475, filed on Feb. 17, 2021.

(51) Int. Cl. C07K 16/28; A61K 39/395; A61K 47/18; A61K 47/26

(52) U.S. Cl. CPC C07K 16/2878; A61K 39/39591; A61K 47/183; A61K 47/26; C07K 2317/31; C07K 2317/622

(57) ABSTRACT

The present disclosure provides pharmaceutical compositions comprising bispecific antibodies and antigen binding fragments thereof that bind to 4-1BB and OX40. Also provided are methods for treating disorders, such as cancer, using such compositions.

### CROSS-REFERENCE TO RELATED APPLICATION

This International Application claims the priority benefit of U.S. Provisional Application No. 63/150,475, filed on Feb. 17, 2021, which is incorporated herein by reference in its entirety.

### REFERENCE TO SEQUENCE LISTING SUBMITTED ELECTRONICALLY

The content of the electronically submitted sequence listing (Name: 4897_0060000_Seqlisting_ST25.txt; Size: 18,030 bytes; and Date of Creation: Feb. 16, 2022) is herein incorporated by reference in its entirety.

### FIELD OF THE DISCLOSURE

The present disclosure relates to pharmaceutical compositions comprising bispecific antibodies and antigen binding fragments thereof that specifically bind to 4-1BB and OX40. The compositions are useful for the treatment of disorders, including solid tumor cancers. Also provided are methods for treating disorders, such as cancer, using such compositions.

### BACKGROUND

4-1BB (CD137) and OX40 are members of the TNF-receptor (TNFR) family (Bremer, ISRN Oncol.: 371854 (2013)). These receptors are not constitutively present on naïve T or NK cells: their expression is triggered by stimulation of T cells through the T-cell Receptor (TCR), or other stimuli in NK cells. 4-1BB is primarily upregulated in CD8 T cells and NK cells, while OX40 is primarily upregulated on CD4 T cells. The function of these receptors is to provide a co-stimulatory signal to T and NK cells. Activation of these receptors is naturally triggered by trimerization through interaction with 4-1BB Ligand (4-1BBL) or OX40 Ligand (OX40L) trimers, leading to signal transduction and initiation of specific cellular functions. 4-1BB enhances the effector function of CD8 T cells and NK cells through increased expression of IFN-7, granzymes, and anti-apoptotic genes leading to the generation of more and better effector CD8 T and NK cells. OX40 enhances the effector function of CD4 T cells by enhancing their ability to produce IL-2 and clonal expansion of memory CD4 T cells.

4-1BB and OX40 are often expressed on tumor infiltrating lymphocytes, and their expression has been used to identify tumor-specific T cells. Human solid tumors are often infiltrated by lymphocytes, mostly CD8+ and CD4+ T cells. The accumulation of tumor infiltrating lymphocytes is often associated with improved survival among patients affected by various malignancies. (Ye et al., OncoImmunology 2: e27184 (2013); Montler et al., Clin Transl Immunology 5:e70 (2016)).

Preclinical results in a variety of induced and spontaneous tumor models suggest that targeting 4-1BB with agonist antibodies can lead to tumor clearance and durable antitumor immunity. Urelumab and utomilumab are agonist anti-4-1BB monoclonal antibodies with ongoing clinical trials for indications including treatment of solid tumors. Despite initial signs of efficacy, clinical development of urelumab has been hampered by inflammatory liver toxicity at doses above 1 mg/kg. Utomilumab is less potent that urelumab, but it has an improved safety profile as compared to urelumab (Chester et al., Blood 131: 39-57 (2018)). A need exists for an efficacious therapeutic that targets 4-1BB that does not cause liver toxicity as observed with urelumab, or other systemic damage.

OX40 agonists have been reported to increase T-cell infiltration into tumors. And OX40 signaling can prevent Treg-mediated suppression of antitumor immune responses. In several preclinical mouse cancer models, including 4T-1 breast cancer, B16 melanoma, Lewis lung carcinoma and several chemically induced sarcomas, injection of an OX40 agonist has resulted in therapeutic responses. (Ohsima et al., J. Immunology 159:3838-3848 (1997); Imura et al., J. Exp. Med. 183:2185-2195 (1996); Maxwell et al., J. Immunology 164:107-112 (2000); Gough et al., J. Immunotherapy 33(8):798-809 (2010)).

In some cases, researchers have generated protein constructs that contain multiple binding domains (>2) against 4-1BB or OX40, or fusions of multiple OX40L and 4-1BBL extracellular domains to induce agonism. In other examples, there are bispecific proteins that contain binding domain(s) to 4-1BB or OX40 and binding domain(s) to a tumor-specific antigen. Binding and clustering via the tumor antigen binding induces clustering and signaling of 4-1BB and OX40. However, none of these constructs are expected to stimulate the function of tumor infiltrating lymphocytes, namely, CD8+ T cells CD4 T+ cells, and NK cells, and to do so with minimal to no off-target activation of effector cells (i.e., activation through binding to FcγR1, FcγRIIa, FcγRIIb, FcγRIIa, and FcγRIIIb). Therefore, there is a need for treatment options that selectively bolster the activity of tumor infiltrating lymphocytes (with minimal to no effect on circulating lymphocytes), e.g., pharmaceutical compositions comprising bispecific antibodies that bind to and stimulate both 4-1BB and OX40.

### SUMMARY

Some aspects of the present disclosure provide a pharmaceutical composition comprising: (a) a bispecific antibody or antigen-binding fragment thereof that specifically binds 4-1BB and OX40; (b) about 5 mM to about 15 mM of a stability promoting buffer; (c) about 25 mM to about 50 mM of a stabilizing amino acid; (d) about 2% to about 8% w/v of a sugar; and (e) about 0.01% to about 0.03% of a surfactant.

In some aspects of the present disclosure, the stability promoting buffer is selected from the group consisting of succinate, histidine, citrate, and glutamate. In some aspects, the stability promoting buffer is present in the composition in an amount of about 5 mM, about 6 mM, about 7 mM, about 8 mM, about 9 mM, about 10 mM, about 11 mM, about 12 mM, about 13 mM, about 14 mM, or about 15 mM. In some aspects, the stability promoting buffer is glutamate. In some aspects, glutamate is present in the composition in a concentration of about 5 mM.

In some aspects of the present disclosure, the stabilizing amino acid is selected from the group consisting of arginine, methionine, leucine, and glycine. In some aspects, the stabilizing amino acid is present in the composition in a concentration of about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, or about 50 mM. In some aspects, the stabilizing amino acid is leucine. In some aspects the leucine is present in the composition in a concentration of about 40 mM.

In some aspects of the present disclosure, the sugar is selected from the group consisting of: sucrose, trehalose, and sorbitol. In some aspects, the sugar is present in the composition in a concentration from about 2% w/v to about 8% w/v. In some aspects, the sugar is present in the composition in a concentration of about 2% w/v, about 3% w/v, about 4% w/v, about 5% w/v, about 6% w/v, about 7% w/v, or about 8% w/v. In some aspects, the sugar is sucrose. In some aspects, the sucrose is present in the composition in a concentration of about 3% w/v.

In some aspects of the present disclosure, the pharmaceutical composition further comprises a surfactant. In some aspects, the surfactant is present in the composition in a concentration of from about 0.01% w/v to about 0.03% w/v. In some aspects, the surfactant is Polysorbate-80. In some aspects, the Polysorbate-80 is present in the composition in a concentration of about 3% w/v.

In some aspects of the present disclosure, the pH of the composition is in a range from about 4.3 to about 4.7. In some aspects, the pH of the composition is about 4.5.

In some aspects of the present disclosure, the 4-1BB×OX40 bispecific antibody or antigen-binding fragment thereof is present in the composition in a concentration of about 10 mg/mL-50 mg/mL.

In some aspects of the present disclosure, the pharmaceutical composition does not comprise sodium chloride (NaCl).

In some aspects of the present disclosure, the 4-1BB×OX40 bispecific antibody or antigen-binding fragment thereof has a purity of about >90%.

In some aspects of the present disclosure, the pharmaceutical composition provided herein comprises: (a) the 4-1BB×OX40 bispecific antibody or antigen-binding fragment thereof in a concentration of about 10 mg/mL; (b) about 10 mM glutamate; (c) about 40 mM leucine; (d) about 3% w/v sucrose; and (e) about 0.02% polysorbate-80; wherein the pH of the composition is from about 4.3 to about 4.7.

In some aspects of the present disclosure, the pharmaceutical composition provided herein comprises: (a) the 4-1BB×OX40 bispecific antibody or antigen-binding fragment thereof in a concentration of 10 mg/mL; (b) 10 mM glutamate; (c) 40 mM leucine; (d) 3% w/v sucrose; and (e) 0.02% polysorbate-80; wherein the pH of the composition is 4.5.

In some aspects of the present disclosure, the pharmaceutical composition provided herein comprises a 4-1BB×OX40 bispecific antibody or antigen-binding fragment thereof comprising a single chain Fv (scFv).

In some aspects of the present disclosure, the pharmaceutical composition provided herein comprises: a 4-1BB×OX40 bispecific antibody or antigen-binding fragment thereof comprising a Fab, Fab′, F(ab′)2, scFv, disulfide linked Fv, or scFv-Fc.

In some aspects of the present disclosure, the pharmaceutical composition provided herein comprises a 4-1BB×OX40 bispecific antibody or antigen-binding fragment thereof comprises a human 4-1BB antigen-binding domain and a human OX40 antigen-binding domain, wherein: (a) the 4-1BB antigen-binding domain comprises a (i) a VH-CDR 1 comprising the amino acid sequence of SEQ ID NO:1; (ii) a VH-CDR2 comprising the amino acid sequence of SEQ ID NO:2; (iii) a VH-CDR3 comprising the amino acid sequence of SEQ ID NO:3; (iv) a light chain variable domain (VL)-CDR1 comprising the amino acid sequence of SEQ ID NO:4; (v) a VL-CDR2 comprising the amino acid sequence of SEQ ID NO:5; and (vi) a VL-CDR3 comprising the amino acid sequence of SEQ ID NO:6; and (b) the OX40 antigen-binding domain comprises a (i) a VH-CDR1 comprising the amino acid sequence of SEQ ID NO:7; (ii) a VH-CDR2 comprising the amino acid sequence of SEQ ID NO:8; (iii) a VH-CDR3 comprising the amino acid sequence of SEQ ID NO:9; (iv) a VL-CDR1 comprising the amino acid sequence of SEQ ID NO:10; (v) a VL-CDR2 comprising the amino acid sequence of SEQ ID NO:11; and (vi) a VL-CDR3 comprising the amino acid sequence of SEQ ID NO:12.

In some aspects, the 4-1BB×OX40 bispecific antibody or antigen-binding fragment thereof comprises a human 4-1BB antigen-binding domain and a human OX40 antigen-binding domain, wherein: (a) the 4-1BB antigen-binding domain comprises a heavy chain variable region comprising the amino acid sequence of SEQ ID NO: 14 and a light chain variable region comprising the amino acid sequence of SEQ ID NO: 15; and (b) the OX40 antigen-binding domain comprises a heavy chain variable region comprising the amino acid sequence of SEQ ID NO: 17 and a light chain variable region comprising the amino acid sequence of SEQ ID NO: 18.

In some aspects, the 4-1BB×OX40 bispecific antibody or antigen-binding fragment thereof comprises a human 4-1BB antigen-binding domain and a human OX40 antigen-binding domain, wherein: (a) the 4-1BB antigen-binding domain comprises a scFv comprising the amino acid sequence of SEQ ID NO: 16; and (b) the OX40 antigen-binding domain comprises a scFv comprising the amino acid sequence of SEQ ID NO: 19.

In some aspects, the 4-1BB×OX40 bispecific antibody or antigen-binding fragment thereof comprises the amino acid sequence of SEQ ID NO: 13.

In some aspects of the present disclosure, the pharmaceutical composition provided herein comprises: (a) 10 mg/mL of a 4-1BB×OX40 bispecific antibody or antigen-binding fragment thereof, wherein the antibody or antigen-binding fragment thereof comprises: (i) a 4-1BB antigen-binding domain comprising a VH-CDR 1 comprising the amino acid sequence of SEQ ID NO:1; a VH-CDR2 comprising the amino acid sequence of SEQ ID NO:2; a VH-CDR3 comprising the amino acid sequence of SEQ ID NO:3; a light chain variable domain (VL)-CDR1 comprising the amino acid sequence of SEQ ID NO:4; a VL-CDR2 comprising the amino acid sequence of SEQ ID NO:5; and a VL-CDR3 comprising the amino acid sequence of SEQ ID NO:6; and an OX40 antigen-binding domain comprising a VH-CDR1 comprising the amino acid sequence of SEQ ID NO:7; a VH-CDR2 comprising the amino acid sequence of SEQ ID NO:8; a VH-CDR3 comprising the amino acid sequence of SEQ ID NO:9; a VL-CDR1 comprising the amino acid sequence of SEQ ID NO:10; a VL-CDR2 comprising the amino acid sequence of SEQ ID NO:11; and a VL-CDR3 comprising the amino acid sequence of SEQ ID NO:12; (ii) a 4-1BB antigen-binding domain comprising a heavy chain variable region comprising the amino acid sequence of SEQ ID NO: 14 and a light chain variable region comprising the amino acid sequence of SEQ ID NO: 15; and a OX40 antigen-binding domain comprising a heavy chain variable region comprising the amino acid sequence of SEQ ID NO: 17 and a light chain variable region comprising the amino acid sequence of SEQ ID NO: 18; (iii) a 4-1BB antigen-binding domain comprising a scFv comprising the amino acid sequence of SEQ ID NO: 16; and an OX40 antigen-binding domain comprising a scFv comprising the amino acid sequence of SEQ ID NO: 19; or (iv) the amino acid sequence of SEQ ID NO: 13; (b) about 5 mM to about 15 mM glutamate; (c) about 25 mM to about 50 mM leucine; (d) about 2% to about 8% w/v sucrose; and (e) about 0.01% to about 0.03% polysorbate-80; wherein the pH of the composition is from about 4.3 to about 4.7.

In some aspects of the present disclosure, the pharmaceutical composition provided herein comprises: (a) 10 mg/mL of a 4-1BB×OX40 bispecific antibody or antigen-binding fragment thereof, wherein the antibody or antigen-binding fragment thereof comprises: (i) a 4-1BB antigen-binding domain comprising a VH-CDR 1 comprising the amino acid sequence of SEQ ID NO:1; a VH-CDR2 comprising the amino acid sequence of SEQ ID NO:2; a VH-CDR3 comprising the amino acid sequence of SEQ ID NO:3; a light chain variable domain (VL)-CDR1 comprising the amino acid sequence of SEQ ID NO:4; a VL-CDR2 comprising the amino acid sequence of SEQ ID NO:5; and a VL-CDR3 comprising the amino acid sequence of SEQ ID NO:7; and (ii) an OX40 antigen-binding domain comprising a VH-CDR1 comprising the amino acid sequence of SEQ ID NO:7; a VH-CDR2 comprising the amino acid sequence of SEQ ID NO:8; a VH-CDR3 comprising the amino acid sequence of SEQ ID NO:9; a VL-CDR1 comprising the amino acid sequence of SEQ ID NO:10; a VL-CDR2 comprising the amino acid sequence of SEQ ID NO:11; and a VL-CDR3 comprising the amino acid sequence of SEQ ID NO:12; (b) 10 mM glutamate; (c) 40 mM leucine; (d) 3% w/v sucrose; and (e) 0.02% polysorbate-80; wherein the pH of the composition is 4.5.

In some aspects, the pharmaceutical composition comprises: (a) the 4-1BB antigen-binding domain comprises a heavy chain variable region comprising the amino acid sequence of SEQ ID NO: 14 and a light chain variable region comprising the amino acid sequence of SEQ ID NO: 15; and (b) the OX40 antigen-binding domain comprises a heavy chain variable region comprising the amino acid sequence of SEQ ID NO: 17 and a light chain variable region comprising the amino acid sequence of SEQ ID NO: 18.

In some aspects of the present disclosure, the pharmaceutical composition provided herein is a liquid.

In some aspects of the pharmaceutical composition provided herein, the human 4-1BB binding domain and the human OX40 binding domain are on the same polypeptide. In some aspects, the human 4-1BB binding domain is N-terminal to the human OX40 binding domain. In some aspects, the human 4-1BB binding domain is C-terminal to the human OX40 binding domain.

In some aspects of the pharmaceutical composition provided herein, the antibody or antigen-binding fragment thereof comprises an immunoglobulin constant region. In aspects, the immunoglobulin constant region comprises immunoglobulin CH2 and CH3 domains of IgG1. In some aspects, the antibody does not contain a CH1 domain. In some aspects, the immunoglobulin constant region comprises a human IgG1 CH2 domain comprising the substitutions E233P, L234A, L235A, G237A, and K322A and a deletion of G236 according to the EU numbering system.

In some aspects, the antibody or antigen-binding fragment thereof comprises a linker between an immunoglobulin constant region and the human 4-1BB binding domain and/or between an immunoglobulin constant region and the human OX40 binding domain. In some aspects, the linker between the immunoglobulin constant region and the human 4-1BB binding domain and/or between the immunoglobulin constant region and the human OX40 binding domain comprises 10-30 amino acids, 15-30 amino acids, or 20-30 amino acids. In some aspects, the linker between the immunoglobulin constant region and the human 4-1BB binding domain or between the immunoglobulin constant region and the human OX40 binding domain comprises the amino acid sequence (Gly4Ser)n, wherein n=1-5 (SEQ ID NO:21), optionally wherein n=1. In some aspects, the antibody comprises a dimer of two polypeptides, each polypeptide comprising in order from amino-terminus to carboxyl-terminus, a first scFv, a hinge region, an immunoglobulin constant region, and a second scFv, wherein (a) the first scFv comprises a human 4-1BB antigen-binding domain, and the second scFv comprises a human OX40 antigen-binding domain or (b) the first scFv comprises a human OX40 antigen-binding domain and the second scFv comprises a human 4-1BB antigen-binding domain. In some aspects, the dimer is a homodimer.

In some aspects of the present disclosure, the pharmaceutical composition provided herein can be stored at 20° C. In some aspects, pharmaceutical composition provided herein has no significant change in pH after storage at 20° C., after about 3 weeks, about 6 weeks, about 3 months, about 6 months, and about 7 months. In some aspects, pharmaceutical composition provided herein has no significant change in the percent High Molecular Weight content (% HMW) after storage at 20° C., after about 3 weeks, about 6 weeks, about 3 months, about 6 months, or about 7 months. In some aspects, the change in the % HMW after storage at 20° C., after about 3 weeks, about 6 weeks, about 3 months, about 6 months, or about 7 months is less than 5%.

In some aspects of the present disclosure, the pharmaceutical composition provided herein has no significant change in the % HMW after one, two, or three freeze/thaw cycles. In some aspects, the change in the % HMW after one, two, or three freeze/thaw cycles is less than 5%. In some aspects, the % HMW is measured by size exclusion ultra pressure liquid chromatography (SE-UPLC).

In some aspects of the present disclosure, the pharmaceutical composition provided herein has no significant change in aggregate formation after storage at 20° C., after about 3 weeks, about 6 weeks, about 3 months, about 6 months, or about 7 months, as measured by size exclusion ultra pressure liquid chromatography (SE-UPLC).

Also provided herein are methods of treating cancer in a subject, comprising administering to the subject an effective amount of the pharmaceutical composition disclosed herein. In some aspects, the cancer is a solid tumor cancer. In some aspects, the cancer is a sarcoma, a carcinoma, or a lymphoma. In some aspects, the cancer is selected from the group consisting of a melanoma, kidney cancer, pancreatic cancer, lung cancer, stomach cancer, colon/intestinal cancer, prostate cancer, ovarian cancer, breast cancer, liver cancer, brain cancer, or a hematological cancer. In some aspects, the subject is a human. In some aspects, the subject expresses 4-1BB and OX40 on tumor infiltrating lymphocytes.

In some aspects of the present disclosure, the pharmaceutical composition provided herein is for use in the treatment of cancer. In some aspects, the pharmaceutical composition provided herein is for use in the treatment of a solid tumor cancer. In some aspects, the solid tumor cancer is a sarcoma, carcinoma, or lymphoma. In some aspects, the cancer is selected from the group consisting of a melanoma, kidney cancer, pancreatic cancer, lung cancer, colon/intestinal cancer, stomach cancer, prostate cancer, ovarian cancer, breast cancer, liver cancer, brain cancer, or a hematological cancer.

### BRIEF DESCRIPTION OF THE FIGURES

FIGS. 1A and 1B show the T_{agg} analysis of FXX01102 in different protein formulations. (See Example 2.)

FIG. 2 shows the results of a B₂₂ analysis performed on FXX01102 formulated at 10 mg/mL in 10 mM glutamate, 8% w/v sucrose, 0.02% w/v polysorbate-80, with or without 100 mM sodium chloride. (See Example 3.)

FIG. 3 shows the results of a B₂₂ analysis comparing the impact of arginine, methionine, and glycine on 10 mg/mL FXX01102 formulated in 10 mM glutamate, 4% w/v sucrose, pH 4.5. (See Example 4.)

FIG. 4 shows the results of a B₂₂ experiment to determine the impact of leucine on FXX01102 formulated in 10 mM glutamate, 4% w/v sucrose, pH 4.5. (See Example 4.)

FIG. 5 shows a comparison of the T_{agg} plots of 10 mg/mL FXX01102 formulated in 10 mM glutamate, 4% w/v sucrose, pH 4.5, with or without 25 mM MgSO₄, lysine or leucine added (See Example 5).

### DETAILED DESCRIPTION

To facilitate an understanding of the present disclosure, a number of terms and phrases are defined below.

### I. TERMINOLOGY

As used herein, the term “4-1BB” refers to mammalian 4-1BB polypeptides including, but not limited to, native 4-1BB polypeptides and isoforms of 4-1BB polypeptides. “4-1BB” encompasses full-length, unprocessed 4-1BB polypeptides as well as forms of 4-1BB polypeptides that result from processing within the cell. As used herein, the term “CD137” should be understood to be interchangeable with the term “4-1BB.” As used herein, the term “human 4-1BB” refers to a polypeptide comprising the amino acid sequence of SEQ ID NO:1. As used herein, the term “cynomolgus 4-1BB” refers to a polypeptide comprising the amino acid sequence of SEQ ID NO:2. A “4-1BB polynucleotide,” “4-1BB nucleotide,” or “4-1BB nucleic acid” refers to a polynucleotide encoding 4-1BB.

As used herein, the term “OX40” refers to mammalian OX40 polypeptides including, but not limited to, native OX40 polypeptides and isoforms of OX40 polypeptides. “OX40” encompasses full-length, unprocessed OX40 polypeptides as well as forms of OX40 polypeptides that result from processing within the cell. As used herein, the term “human OX40” refers to a polypeptide comprising the amino acid sequence of SEQ ID NO:3. As used herein, the term “cynomolgus OX40” refers to a polypeptide comprising the amino acid sequence of SEQ ID NO:4. An “OX40 polynucleotide,” “OX40 nucleotide,” or “OX40 nucleic acid” refers to a polynucleotide encoding OX40.

As used herein, the term “tumor infiltrating lymphocytes” or “TIL” refers to lymphocytes that directly oppose and/or surround tumor cells. Tumor infiltrating lymphocytes are typically non-circulating lymphocytes and include, CD8+ T cells, CD4+ T cells and NK cells. Tumor infiltrating lymphocytes can express OX40 and 4-1BB.

As used herein, the terms “antibody” and “antibodies” are terms of art and can be used interchangeably herein and refer to a molecule or a complex of molecules with at least one antigen-binding site that specifically binds an antigen.

Antibodies can include, for example, monoclonal antibodies, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), bispecific antibodies, and multi-specific antibodies. In some aspects, antibodies described herein refer to polyclonal antibody populations. Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂ₐ or IgG_{2b}) of immunoglobulin molecule. In some aspects, antibodies described herein are IgG antibodies, or a class (e.g., human IgG₁, IgG₂, or IgG₄) or subclass thereof. In a specific aspect, the antibody is a humanized monoclonal antibody. In another specific aspect, the antibody is a human monoclonal antibody, e.g., that is an immunoglobulin. In some aspects, an antibody described herein is an IgG₁, IgG₂, or IgG₄ antibody.

“Bispecific” antibodies are antibodies with two different antigen-binding sites (exclusive of the Fc region) that bind to two different antigens. Bispecific antibodies can include, for example, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, heteroconjugate antibodies, linked single chain antibodies or linked-single-chain Fvs (scFv), camelized antibodies, affybodies, linked Fab fragments, F(ab′)₂ fragments, chemically-linked Fvs, and disulfide-linked Fvs (sdFv). Bispecific antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂ₐ or IgG_{2b}) of immunoglobulin molecule. In some aspects, bispecific antibodies described herein are IgG antibodies, or a class (e.g., human IgG₁, IgG₂, or IgG₄) or subclass thereof. In some aspects, bispecific antibodies described herein comprise two polypeptides, optionally identical polypeptides, each polypeptide comprising in order from amino-terminus to carboxyl-terminus, a first scFv antigen-binding domain, a linker (optionally wherein the linker is a hinge region), an immunoglobulin constant region, and a second scFv antigen-binding domain. This particular type of antibody is exemplified by ADAPTIR™ technology. An exemplary molecule is referred to throughout the present disclosure as FXX01102. Bispecific antibodies can be e.g., monovalent for each target (e.g., an IgG molecule with one arm targeting one antigen and the other arm targeting a second antigen) or bivalent for each target (e.g., a dual variable domain antibody, an IgG-scFv, a scFv-Fc-scFv, or an ADAPTIR™ antibody containing a dimer, wherein each polypeptide of the dimer contains two different antigen-binding domains).

As used herein, the terms “antigen-binding domain,” “antigen-binding region,” “antigen-binding site,” and similar terms refer to the portion of antibody molecules which comprises the amino acid residues that confer on the antibody molecule its specificity for the antigen (e.g., the complementarity determining regions (CDR)). The antigen-binding region can be derived from any animal species, such as rodents (e.g., mouse, rat, or hamster) and humans. An antigen-binding domain that binds to 4-1BB can be referred to herein e.g., as a “4-1BB binding domain.” An antigen-binding domain that binds to OX40 can be referred to herein e.g., as an “OX40 binding domain.” As used herein, a “human 4-1BB binding domain” or “human 4-1BB antigen-binding domain” refers to an antigen-binding domain that specifically binds to human 4-1BB although it may also bind to a non-human 4-1BB (for instance, murine, rodent, or non-human primate 4-1BB). Likewise, a “human OX40 binding domain” or “human OX40 antigen-binding domain” refers to an antigen-binding domain that specifically binds to human OX40.

A used herein, the term “4-1BB/OX40 antibody,” “anti-4-1BB/OX40 antibody” or “4-1BB×OX40 antibody” refers to a bispecific antibody that contains an antigen-binding domain that binds to 4-1BB (e.g., human 4-1BB) and an antigen-binding domain that binds to OX40 (e.g., human OX40).

A “monoclonal” antibody refers to a homogeneous antibody population involved in the highly specific recognition and binding of a single antigenic determinant, or epitope. This is in contrast to polyclonal antibodies that typically include different antibodies directed against different antigenic determinants. The term “monoclonal” antibody encompasses both intact and full-length immunoglobulin molecules as well Fab, Fab′, F(ab′)2, Fv), single chain (scFv), fusion proteins comprising an antibody portion, and any other modified immunoglobulin molecule comprising an antigen recognition site. Furthermore, a “monoclonal” antibody refers to such antibodies made in any number of manners including but not limited to by hybridoma, phage selection, recombinant expression, and transgenic animals.

The term “chimeric” antibodies refers to antibodies wherein the amino acid sequence is derived from two or more species. Typically, the variable region of both light and heavy chains corresponds to the variable region of antibodies derived from one species of mammals (e.g. mouse, rat, rabbit, etc.) with the desired specificity, affinity, and capability while the constant regions are homologous to the sequences in antibodies derived from another (usually human) to avoid eliciting an immune response in that species.

The term “humanized” antibody refers to forms of non-human (e.g. murine) antibodies that contain minimal non-human (e.g., murine) sequences. Typically, humanized antibodies are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody from a non-human species that has the desired specificity, affinity, and capability. The humanized antibody thereof can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996).

The term “human” antibody means an antibody having an amino acid sequence derived from a human immunoglobulin gene locus, where such antibody is made using any technique known in the art.

The variable region typically refers to a portion of an antibody, generally, a portion of a light or heavy chain, typically about the amino-terminal 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some aspects, the variable region is a human variable region. In some aspects, the variable region comprises rodent or murine CDRs and human framework regions (FRs). In particular aspects, the variable region is a primate (e.g., non-human primate) variable region. In some aspects, the variable region comprises rodent or murine CDRs and primate (e.g., non-human primate) framework regions (FRs).

The terms “VH” and “VH domain” are used interchangeably to refer to the heavy chain variable region of an antibody.

The terms “VL” and “VL domain” are used interchangeably to refer to the light chain variable region of an antibody.

The term “Kabat numbering” and like terms are recognized in the art and refer to a system of numbering amino acid residues in the heavy and light chain variable regions of an antibody, or an antigen-binding portion thereof. In some aspects, the CDRs of an antibody can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In a specific embodiment, the CDRs of the antibodies described herein have been determined according to the Kabat numbering scheme.

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). In a specific embodiment, the CDRs of the antibodies described herein have been determined according to the Chothia numbering scheme.

The AbM hypervariable regions represent a compromise between the Kabat CDRs and Chothia structural loops, and are used by Oxford Molecular's AbM antibody modeling software. In a specific embodiment, the CDRs of the antibodies described herein have been determined according to the AbM numbering scheme.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The IMGT numbering convention is described in Brochet, X, et al, Nucl. Acids Res. 36: W503-508 (2008). In a specific embodiment, the CDRs of the antibodies described herein have been determined according to the IMGT numbering convention. As used herein, unless otherwise provided, a position of an amino acid residue in a variable region of an immunoglobulin molecule is numbered according to the IMGT numbering convention.

As used herein, the term “constant region” or “constant domain” are interchangeable and have its meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. An immunoglobulin “constant region” or “constant domain” can contain a CH1 domain, a hinge, a CH2 domain, and a CH3 domain or a subset of these domains, e.g., a CH2 domain and a CH3 domain. In some aspects provided herein, an immunoglobulin constant region does not contain a CH1 domain. In some aspects provided herein, an immunoglobulin constant region does not contain a hinge. In some aspects provided herein, an immunoglobulin constant region contains a CH2 domain and a CH3 domain.

“Fc region” or “Fc domain” refers to a polypeptide sequence corresponding to or derived from the portion of a source antibody that is responsible for binding to antibody receptors on cells and the C1q component of complement. Fc stands for “fragment crystalline,” and refers to the fragment of an antibody that will readily form a protein crystal. Distinct protein fragments, which were originally described by proteolytic digestion, can define the overall general structure of an immunoglobulin protein. An “Fc region” or “Fc domain” contains a CH2 domain, a CH3 domain, and optionally all or a portion of a hinge. An “Fc region” or “Fc domain” can refer to a single polypeptide or to two disulfide-linked polypeptides. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc includes variants of naturally occurring sequences.

An “immunoglobulin dimerization domain” or “immunoglobulin heterodimerization domain,” as used herein, refers to an immunoglobulin domain of a polypeptide chain that preferentially interacts or associates with a different immunoglobulin domain of a second polypeptide chain, wherein the interaction of the different immunoglobulin heterodimerization domains substantially contributes to or efficiently promotes heterodimerization of the first and second polypeptide chains (i.e., the formation of a dimer between two different polypeptide chains, which is also referred to as a “heterodimer”). The interactions between immunoglobulin heterodimerization domains “substantially contributes to or efficiently promotes” the heterodimerization of first and second polypeptide chains if there is a statistically significant reduction in the dimerization between the first and second polypeptide chains in the absence of the immunoglobulin heterodimerization domain of the first polypeptide chain and/or the immunoglobulin heterodimerization domain of the second polypeptide chain. In some aspects, when the first and second polypeptide chains are co-expressed, at least 60%, at least about 60% to about 70%, at least about 70% to about 80%, at least 80% to about 90%, 91%, 92%, 93%, 94%, 95%, 96%, 97%, 98%, or 99% of the first and second polypeptide chains form heterodimers with each other. Representative immunoglobulin heterodimerization domains include an immunoglobulin CH1 domain, an immunoglobulin CL domain (e.g., Cκ or Cλ isotypes), or derivatives thereof, including wild type immunoglobulin CH1 and CL domains and altered (or mutated) immunoglobulin CH1 and CL domains, as provided therein.

A “wild-type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of a naturally occurring antibody. In some aspects, a wild type immunoglobulin hinge region sequence is human, and can comprise a human IgG hinge region. An “altered wild-type immunoglobulin hinge region” or “altered immunoglobulin hinge region” refers to (a) a wild type immunoglobulin hinge region with up to 30% amino acid changes (e.g., up to 25%, 20%, 15%, 10%, or 5% amino acid substitutions or deletions), or (b) a portion of a wild type immunoglobulin hinge region that has a length of about 5 amino acids (e.g., about 5, 6, 7, 8, 9, 10, 11, 12, 13, 14, 15, 16, 17, 18, 19, or 20 amino acids) up to about 120 amino acids (for instance, having a length of about 10 to about 40 amino acids or about 15 to about 30 amino acids or about 15 to about 20 amino acids or about 20 to about 25 amino acids), has up to about 30% amino acid changes (e.g., up to about 25%, 20%, 15%, 10%, 5%, 4%, 3%, 2%, or 1% amino acid substitutions or deletions or a combination thereof), and has an IgG core hinge region as disclosed in US 2013/0129723 and US 2013/0095097. As provided herein, a “hinge region” or a “hinge” can be located between an antigen-binding domain (e.g., a 4-1BB or an OX40-binding domain) and an immunoglobulin constant region.

As used herein, a “linker” refers to a moiety, e.g., a polypeptide, that is capable of joining two compounds, e.g., two polypeptides. Non-limiting examples of linkers include flexible linkers comprising glycine-serine (e.g., (Gly4Ser)) repeats, and linkers derived from (a) an interdomain region of a transmembrane protein (e.g., a type I transmembrane protein); (b) a stalk region of a type II C-lectin; or (c) an immunoglobulin hinge. As provided herein, a linker can refer, e.g., to (1) a polypeptide region between VH and VL regions in a single-chain Fv (scFv) or (2) a polypeptide region between an immunoglobulin constant region and an antigen-binding domain. In some aspects, a linker is comprised of 5 to about 35 amino acids, for instance, about 15 to about 25 amino acids. In some aspects, a linker is comprised of at least 5 amino acids, at least 7 amino acids or at least 9 amino acids.

As used herein, the term “heavy chain” when used in reference to an antibody can refer to any distinct type, e.g., alpha (α), delta (δ), epsilon (ε), gamma (γ), and mu (μ), based on the amino acid sequence of the constant region, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (κ) or lambda (λ) based on the amino acid sequence of the constant regions. Light chain amino acid sequences are well known in the art. In specific aspects, the light chain is a human light chain.

As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991, each of which is herein incorporated by reference in its entirety. As used herein, unless otherwise provided, a position of an amino acid residue in a constant region of an immunoglobulin molecule is numbered according to EU nomenclature (Ward et al., 1995 Therap. Immunol. 2:77-94).

As used herein, the term “dimer” refers to a biological entity that consists of two subunits associated with each other via one or more forms of intramolecular forces, including covalent bonds (e.g., disulfide bonds) and other interactions (e.g., electrostatic interactions, salt bridges, hydrogen bonding, and hydrophobic interactions), and is stable under appropriate conditions (e.g., under physiological conditions, in an aqueous solution suitable for expressing, purifying, and/or storing recombinant proteins, or under conditions for non-denaturing and/or non-reducing electrophoresis). A “heterodimer” or “heterodimeric protein,” as used herein, refers to a dimer formed from two different polypeptides. A “homodimer” or “homodimeric protein,” as used herein, refers to a dimer formed from two identical polypeptides.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody to an antigen, and k_{off} refers to the dissociation of, e.g., an antibody from an antigen. The kₒₙ and k_{off} can be determined by techniques known to one of ordinary skill in the art, such as BIAcore® or KinExA.

As used herein, the terms “immunospecifically binds,” “immunospecifically recognizes,” “specifically binds,” and “specifically recognizes” are analogous terms in the context of antibodies. These terms indicate that the antibody binds to an epitope via its antigen-binding domain and that the binding entails some complementarity between the antigen-binding domain and the epitope. Accordingly, an antibody that “specifically binds” to human 4-1BB and/or OX40 may also, but the extent of binding to an un-related, non-4-1BB and/or OX40 protein is less than about 10% of the binding of the antibody to 4-1BB and/or OX40 as measured, e.g., by a radioimmunoassay (RIA).

Binding domains can be classified as “high affinity” binding domains and “low affinity” binding domains. “High affinity” binding domains refer to those binding domains with a K_{D} value less than 10⁻⁷ M, less than 10⁻⁸ M, less than 10⁻⁹ M, less than 10⁻¹⁰ M. “Low affinity” binding domains refer to those binding domains with a KD greater than 10⁻⁷ M, greater than 10⁻⁶ M, or greater than 10⁻⁵ M. “High affinity” and “low affinity” binding domains bind their targets, while not significantly binding other components present in a test sample.

As used herein, an antibody is “capable of binding” if it will specifically bind its target (i.e., human 4-1BB or human OX40) when in close proximity to the target and under conditions one of skill in the art would consider to be necessary for binding. A “human 4-1BB antigen-binding domain” should be understood to mean a binding domain that specifically binds to human 4-1BB. A “human OX40 antigen-binding domain” should be understood to mean a binding domain that specifically binds to OX40.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in some aspects, the polypeptides can occur as single chains or associated chains.

The terms “pharmaceutical formulation” and “pharmaceutical composition” refer to a preparation which is in such form as to permit the biological activity of the active ingredient to be effective, and which contains no additional components which are unacceptably toxic to a subject to which the formulation would be administered. The formulation can be sterile.

As used herein, the term “pharmaceutically acceptable” refers to molecular entities and compositions that do not generally produce allergic or other serious adverse reactions when administered using routes well known in the art. Molecular entities and compositions approved by a regulatory agency of the Federal or a state government or listed in the U.S. Pharmacopeia or other generally recognized pharmacopeia for use in animals, and more particularly in humans are considered to be “pharmaceutically acceptable.”

The terms “administer”, “administering”, “administration”, and the like, as used herein, refer to methods that may be used to enable delivery of a drug, e.g., a 4-1BB/OX40 antibody or antigen binding fragment thereof to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

As used herein, the terms “subject” and “patient” are used interchangeably. The subject can be an animal. In some aspects, the subject is a mammal such as a non-human animal (e.g., cow, pig, horse, cat, dog, rat, mouse, monkey or other primate, etc.). In some aspects, the subject is a human. As used herein, the term “patient in need” or “subject in need” refers to a patient at risk of, or suffering from, a disease, disorder or condition that is amenable to treatment or amelioration, e.g., with a 4-1BB/OX40 antibody or antigen binding fragment thereof provided herein. A patient in need may, for instance, be a patient diagnosed with a cancer.

The term “therapeutically effective amount” refers to an amount of a drug, e.g., an anti-4-1BB/OX40 antibody effective to treat a disease or disorder in a subject. In the case of cancer, the therapeutically effective amount of the drug can reduce the number of cancer cells; reduce the tumor size or burden; inhibit (i.e., slow to some extent and in a certain aspect, stop) cancer cell infiltration into peripheral organs; inhibit (i.e., slow to some extent and in a certain aspect, stop) tumor metastasis; inhibit, to some extent, tumor growth; relieve to some extent one or more of the symptoms associated with the cancer; and/or result in a favorable response such as increased progression-free survival (PFS), disease-free survival (DFS), or overall survival (OS), complete response (CR), partial response (PR), or, in some cases, stable disease (SD), a decrease in progressive disease (PD), a reduced time to progression (TTP), or any combination thereof.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. In some aspects, a subject is successfully “treated” for cancer according to the methods of the present invention if the patient shows one or more of the following: a reduction in the number of or complete absence of cancer cells; a reduction in the tumor size; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; increased progression-free survival (PFS), disease-free survival (DFS), or overall survival (OS), complete response (CR), partial response (PR), stable disease (SD), a decrease in progressive disease (PD), a reduced time to progression (TTP), or any combination thereof.

The terms “cancer” and “cancerous” refer to or describe the physiological condition in mammals in which a population of cells are characterized by unregulated cell growth. Examples of cancer include, but are not limited to, melanoma, kidney cancer, pancreatic cancer, lung cancer, intestinal cancer, prostate cancer, breast cancer, liver cancer, brain cancer, and hematological cancers. The cancer may be a primary tumor or may be advanced or metastatic cancer.

A cancer can be a solid tumor cancer. The term “solid tumor” refers to an abnormal mass of tissue that usually does not contain cysts or liquid areas. Examples of solid tumors are sarcomas, carcinomas, and lymphomas. Leukemias (cancers of the blood) generally do not form solid tumors. A solid tumor can contain tumor infiltrating lymphocytes which express OX40 and 4-1BB.

It should be understood that the terms “a” and “an” as used herein refer to “one or more” of the enumerated components unless otherwise indicated.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided and part of the present application's disclosure. In this disclosure, “comprises,” “comprising,” “containing” and “having” and the like can have the meaning ascribed to them in U.S. and European Patent law and can mean “includes,” “including,” and the like; “consisting essentially of” or “consists essentially” likewise has the meaning ascribed in U.S. and European Patent law. It should be appreciated that as far as U.S. patent law is concerned, the term is open-ended, allowing for the presence of more than that which is recited so long as basic or novel characteristics of that which is recited is not changed by the presence of more than that which is recited, but excludes prior art aspects. It should also be appreciated that as far as European Patent law is concerned the use of “consisting essentially of” or “comprising substantially” means that specific further components can be present, namely those not materially affecting the essential characteristics of the compound or composition.

As used herein, the terms “about” and “approximately,” when used to modify a numeric value or numeric range, indicate that deviations of up to 5% above or 5% below the value or range remain within the intended meaning of the recited value or range.

Any domains, components, compositions, and/or methods provided herein can be combined with one or more of any of the other domains, components, compositions, and/or methods provided herein.

### II. PHARMACEUTICAL COMPOSITIONS

This disclosure provides novel pharmaceutical compositions comprising 4-1BB×OX40 bispecific antibodies or antigen-binding fragments thereof, having enhanced stability relative to known formulations. The novel pharmaceutical compositions provided herein inhibit aggregate formation over time during storage at 20° C., as well as after multiple freeze thaw cycles. The novel pharmaceutical compositions of the disclosure include 4-1BB×OX40 bispecific antibodies or antigen-binding fragments thereof, formulated with particular excipients (i.e., a stability promoting buffer, a stabilizing amino acid, a sugar component, and surfactant) for improved stability. Advantageously, the novel pharmaceutical compositions provide enhanced stability over a range of pH values.

Solution conditions that stabilize a protein's secondary and tertiary structure in order to minimize the formation of aggregate or degradation products are desirable for therapeutic formulations. For therapeutic proteins used to treat disease, e.g., 4-1BB×OX40 bispecific antibodies or antigen-binding fragments thereof, product-related contaminants can have undesirable consequences, including loss of potency, unexpected increase in potency, immunogenicity and other unwanted side effects. A formulation can consist of several components and is typically provided in an aqueous solution. Such components can include but are not limited to: a buffer to maintain a certain pH and to resist changes in pH during storage, salts, amino acids, detergents, polymers, sugars, and other chemical excipients, such as surfactants, that serve to maintain the stability or solubility of the drug, or to incorporate other desirable properties to the solution. Such components could have positive or negative charges, could be zwitterions or could be amphiphilic, hydrophobic or hydrophilic. Different components may bind, interact and/or stabilize different regions of the protein based on their chemical characteristics. Pharmaceutical compositions suitable for administration to human patients are typically formulated for parenteral administration, e.g., in a liquid carrier, or suitable for reconstitution into liquid solution or suspension for intravenous or subcutaneous administration. Liquid compositions for parenteral administration can be formulated for administration by injection or continuous infusion. Routes of administration by injection or infusion include intravenous, intraperitoneal, intramuscular, intrathecal and subcutaneous. In some aspects, the pharmaceutical formulations disclosed herein are lyophilized prior to administration. In some aspects, the pharmaceutical formulations disclosed herein are not lyophilized prior to administration.

The present disclosure provides novel pharmaceutical compositions comprising 4-1BB×OX40 bispecific antibodies or antigen-binding fragments thereof. In some aspects, the disclosure provides (a) a bispecific antibody or antigen-binding fragment thereof that specifically binds 4-1BB and OX40; (b) about 5 mM to about 15 mM of a stability promoting buffer; (c) about 25 mM to about 50 mM of a stabilizing amino acid; (d) about 2% to about 8% w/v of a sugar; and (e) about 0.01% to about 0.03% of a surfactant.

In some aspects, the pharmaceutical compositions comprising 4-1BB×OX40 bispecific antibodies or antigen-binding fragments thereof disclosed herein comprise a stability promoting buffer selected from the group consisting of succinate, histidine, citrate, and glutamate. In some aspects, the stability promoting buffer is present in the composition in a concentration of about 5 mM, about 6 mM, about 7 mM, about 8 mM, about 9 mM, about 10 mM, about 11 mM, about 12 mM, about 13 mM, about 14 mM, or about 15 mM. In some aspects, the stability promoting buffer is glutamate. In some aspects, the glutamate is present in the composition in a concentration of about 5 mM.

In some aspects, the pharmaceutical compositions comprising 4-1BB×OX40 bispecific antibodies or antigen-binding fragments thereof disclosed herein comprise a stabilizing amino acid selected from the group consisting of arginine, methionine, leucine, and glycine. In some aspects, the stabilizing amino acid is present in the composition in a concentration of about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, or about 50 mM. In some aspects, the stabilizing amino acid is leucine. In some aspects, the leucine is present in the composition in a concentration of about 40 mM.

In some aspects, the pharmaceutical compositions comprising 4-1BB×OX40 bispecific antibodies or antigen-binding fragments thereof disclosed herein comprises a sugar selected from the group consisting of: sucrose, trehalose, and sorbitol. In some aspects, the sugar is present in the composition in a concentration from about 2% w/v to about 8% w/v. In some aspects, the sugar is present in the composition in a concentration of about 2% w/v, about 3% w/v, about 4% w/v, about 5% w/v, about 6% w/v, about 7% w/v, or about 8% w/v. In some aspects, the sugar is sucrose. In some aspects, the sucrose is present in the composition in a concentration of about 3% w/v.

In some aspects, the pharmaceutical compositions comprising 4-1BB×OX40 bispecific antibodies or antigen-binding fragments thereof disclosed herein comprise a surfactant. In some aspects, the surfactant is present in the composition in a concentration of from about 0.01% w/v to about 0.03% w/v. In some aspects, the surfactant is Polysorbate-80. In some aspects, the Polysorbate-80 is present in the composition in a concentration of about 3% w/v.

In some aspects, the pharmaceutical compositions comprising 4-1BB×OX40 bispecific antibodies or antigen-binding fragments thereof disclosed herein provide enhanced stability within a certain pH range. In some aspects, the pH of the composition is in a range from about 4.3 to about 4.7. In some aspects, the pH of the composition is about 4.5. In some aspects, the pH of the composition is 4.5.

In some aspects of the pharmaceutical compositions disclosed herein, the 4-1BB×OX40 bispecific antibody or antigen-binding fragment thereof is present in the composition in a concentration of about 10-50 mg/mL. In some aspects, the 4-1BB×OX40 bispecific antibody or antigen-binding fragment thereof is present in the composition in a concentration of about 10 mg/mL. In some aspects, the 4-1BB×OX40 bispecific antibody or antigen-binding fragment thereof is present in the composition in a concentration of about 20 mg/mL. In some aspects the 4-1BB×OX40 bispecific antibody or antigen-binding fragment thereof is present in the composition in a concentration of about 30 mg/mL. In some aspects the 4-1BB×OX40 bispecific antibody or antigen-binding fragment thereof is present in the composition in a concentration of about 40 mg/mL. In some aspects the 4-1BB×OX40 bispecific antibody or antigen-binding fragment thereof is present in the composition in a concentration of about 50 mg/mL.

The addition of sodium chloride (NaCl) to a pharmaceutical composition comprising an 4-1BB×OX40 bispecific antibody or antigen-binding fragment thereof formulated with glutamate, sucrose, and Polysorbate-80 can have a destabilizing effect on the composition and can promote protein-protein interactions that could lead to protein aggregation. Accordingly, in some aspects, the pharmaceutical composition disclosed herein does not comprise sodium chloride (NaCl).

In some aspects of the pharmaceutical compositions disclosed herein, the 4-1BB×OX40 bispecific antibody or antigen-binding fragment thereof has a purity of about >90%. In some aspects, the 4-1BB×OX40 bispecific antibody or antigen-binding fragment thereof has a purity of about >91%. In some aspects, the 4-1BB×OX40 bispecific antibody or antigen-binding fragment thereof has a purity of about >92%. In some aspects, the 4-1BB×OX40 bispecific antibody or antigen-binding fragment thereof has a purity of about >93%. In some aspects, the 4-1BB×OX40 bispecific antibody or antigen-binding fragment thereof has a purity of about >94%. In some aspects, the 4-1BB×OX40 bispecific antibody or antigen-binding fragment thereof has a purity of about >95%.

In some aspects, provided herein are pharmaceutical compositions comprising: (a) the 4-1BB×OX40 bispecific antibody or antigen-binding fragment thereof in a concentration of about 10 mg/mL; (b) about 10 mM glutamate; (c) about 40 mM leucine; (d) about 3% w/v sucrose; and (e) about 0.02% polysorbate-80; wherein the pH of the composition is from about 4.3 to about 4.7.

In some aspects, provided herein are pharmaceutical compositions comprising: comprising: (a) the 4-1BB×OX40 bispecific antibody or antigen-binding fragment thereof in a concentration of 10 mg/mL; (b) 10 mM glutamate; (c) 40 mM leucine; (d) 3% w/v sucrose; and (e) 0.02% polysorbate-80; wherein the pH of the composition is 4.5.

In general, such compositions typically can further comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. Such pharmaceutical carriers include but are not limited to: sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions can be employed as carriers, particularly for injectable solutions.

### III. 4-1BB×OX40 BISPECIFIC ANTIBODIES

Provided herein are novel pharmaceutical compositions comprising 4-1BB×OX40 bispecific antibodies or antigen-binding fragments thereof comprising an antigen-binding domain that specifically binds to human 4-1BB (i.e., a human 4-1BB antigen-binding domain) and an antigen-binding domain that binds to human OX40 (i.e., a human OX40 antigen-binding domain). The 4-1BB×OX40 bispecific antibodies or antigen-binding fragments thereof described herein are bivalent for both target proteins, i.e., the 4-1BB×OX40 bispecific antibodies or antigen-binding fragments thereof contain two 4-1BB binding domains and two OX40 binding domains. Such 4-1BB×OX40 bispecific antibodies or antigen-binding fragment thereof are exemplified by ADAPTIR™ technology. An exemplary molecule is referred to throughout the present disclosure as FXX01102. Bispecific antibodies or antigen-binding fragments thereof that are bivalent for each target include but are not limited to: a dual variable domain antibody, an IgG-scFv, a scFv-Fc-scFv, or an ADAPTIR™ antibody containing a dimer, wherein each polypeptide of the dimer contains two different antigen-binding domains. In some aspects, the 4-1BB×OX40 bispecific antibodies or antigen-binding fragments thereof described herein comprise a single chain Fv (scFv). In some aspects, the 4-1BB×OX40 bispecific antibodies or antigen-binding fragments thereof described herein comprise a Fab, Fab′, F(ab′)2, scFv, disulfide linked Fv, or scFv-Fc.

A. 4-1BB Binding Domains

Provided herein are antigen-binding domains that bind to human 4-1BB (i.e., 4-1BB binding domains) that can be used to assemble 4-1BB×OX40 bispecific antibodies or antigen binding fragments thereof. A 4-1BB binding domain can bind to 4-1BB from other species, e.g. cynomolgus monkey and/or mouse 4-1BB, in addition to binding to human 4-1BB. In some aspects, the 4-1BB binding domains bind to human 4-1BB and to cynomolgus monkey 4-1BB.

A 4-1BB binding domain can comprise six complementarity determining regions (CDRs), i.e., a variable heavy chain (VH) CDR1, a VH CDR2, a VH CDR3, a variable light chain (VL) CDR1, a VL CDR2, and a VL CDR3. A 4-1BB binding domain can comprise a variable heavy chain (VH) and a variable light chain (VL). The VH and the VL can be separate polypeptides or can be on the same polypeptide (e.g., in an scFv).

In some aspects, a 4-1BB binding domain described herein comprises a combination of six CDRs provided in Table A (e.g., SEQ ID NOs:1-6).

TABLE A
4-1BB VH CDR Amino Acid Sequences¹

| VH CDR1 | VH CDR2 | VH CDR3 |
|---|---|---|
| (SEQ ID NO:) | (SEQ ID NO:) | (SEQ ID NO:) |
| GYTFTSYW | IYPSGGST | ASFSDGYYAYAMDY |
| (SEQ ID NO: 1) | (SEQ ID NO: 2) | (SEQ ID NO: 3) |

¹The CDRs are determined according to IMGT.

TABLE B
4-1BB VL CDR Amino Acid Sequences⁴

| VL CDR1 | VL CDR2 | VL CDR3 |
|---|---|---|
| (SEQ ID NO:) | (SEQ ID NO:) | (SEQ ID NO:) |
| QDISNY | YTS | QQGYTLPYT |
| (SEQ ID NO: 4) | (SEQ ID NO: 5) | (SEQ ID NO: 6) |

⁴The CDRs are determined according to IMGT.

A 4-1BB×OX40 bispecific antibody or antigen binding fragment thereof that is bivalent for 4-1BB can comprise two 4-1BB binding domains, each comprising the six CDRs listed in Tables A and B above.

In some aspects of the 4-1BB×OX40 bispecific antibody or antigen binding fragment thereof described herein, a 4-1BB binding domain comprises the VH sequence provided in Table C.

TABLE C
4-1BB Variable Heavy Chain (VH)
Amino Acid Sequence

| SEQ ID NO | VH Amino Acid Sequence |
|---|---|
| 14 | EVQLVQSGAEVKKPGASVKVSCKASGYTFTSYWMNWVR |
| | QAPGQGLEWMGNIYPSGGSTNYAQKFQGRVTMTVDTST |
| | STVYMELSSLRSEDTAVYYCASFSDGYYAYAMDYWGQG |
| | TLVTVSS |

A 4-1BB binding domain described herein can comprise a VH comprising the CDRs of a VH sequence in Table C, e.g., the IMGT-defined CDRs, the Kabat-defined CDRs, the Chothia-defined CDRs, or the AbM-defined CDRs.

In some aspects of the 4-1BB×OX40 bispecific antibody or antigen binding fragment thereof described herein, a 4-1BB binding domain comprises the VL sequence provided in Table D.

TABLE D
4-1BB Variable Light Chain (VL)
Amino Acid Sequence

| SEQ ID NO | VL Amino Acid Sequence |
|---|---|
| 15 | EIVMTQSPATLSLSPGERATLSCRASQSVSSYLNWYQQ |
| | KPGQAPRLLIYYASRRHTGIPARFSGSGSGTDFTLTIS |
| | SLQPEDFAVYYCQQGYNLPYTFGQGTKVEIK |

A 4-1BB binding domain described herein can comprise a VL comprising the CDRs of a VL sequence in Table D, e.g., the IMGT-defined CDRs, the Kabat-defined CDRs, the Chothia-defined CDRs, or the AbM-defined CDRs.

In some aspects, the 4-1BB×OX40 bispecific antibody or antigen binding fragment thereof described herein can comprise two 4-1BB binding domains, each comprising a VH provided in Table C and a VL provided in Table D. The VH provided in Table C and the VL provided in table D can be on different polypeptides or can be on the same polypeptide. When the VH and VL are on the same polypeptide, they can be in either orientation (i.e., VH-VL or VL-VH), and they can be connected by a linker (e.g., a glycine-serine linker). In some aspects, the VH and VL are connected a glycine-serine linker that is at least 15 amino acids in length (e.g., 15-50 amino acids 15-40 amino acids, 15-30 amino acids, 15-25 amino acids or 15-20 amino acids). In some aspects, the VH and VL are connected a glycine-serine linker that is at least 20 amino acids in length (e.g., 20-50 amino acids 20-40 amino acids, 20-30 amino acids, or 20-25 amino acids).

In some aspects of the 4-1BB×OX40 bispecific antibody or antigen binding fragment described herein, a 4-1BB binding domain can comprise a VH comprising the CDRs of a VH sequence in Table C, e.g., the IMGT-defined CDRs, the Kabat-defined CDRs, the Chothia-defined CDRs, or the AbM-defined CDRs and a VL comprising the CDRs of a VL sequence in Table D, e.g., the IMGT-defined CDRs, the Kabat-defined CDRs, the Chothia-defined CDRs, or the AbM-defined CDRs.

B. OX40 Binding Domains

Provided herein are antigen-binding domains that bind to human OX40 (i.e., OX40 binding domains) that can be used to assemble 4-1BB×OX40 bispecific antibodies or antigen binding fragments thereof. An OX40 binding domain can bind to OX40 from other species, e.g. cynomolgus monkey and/or mouse OX40, in addition to binding to human OX40. In some aspects, the OX40 binding domains bind to human OX40 and to cynomolgus monkey OX40.

An OX40 binding domain can comprise six complementarity determining regions (CDRs), i.e., a variable heavy chain (VH) CDR1, a VH CDR2, a VH CDR3, a variable light chain (VL) CDR1, a VL CDR2, and a VL CDR3. An OX40 binding domain can comprise a variable heavy chain (VH) and a variable light chain (VL). The VH and the VL can be separate polypeptides or can be on the same polypeptide (e.g., in an scFv).

In some aspects, an OX40 binding domain described herein comprises the six CDRs listed in Tables E and F.

TABLE E
OX40 VH CDR Amino Acid Sequences³

| VH CDR1 | VH CDR2 | VH CDR3 |
|---|---|---|
| (SEQ ID NO:) | (SEQ ID NO:) | (SEQ ID NO:) |
| GFTLSYYG | ISHDGSDK | SNDQFDP |
| (SEQ ID NO: 7) | (SEQ ID NO: 8) | (SEQ ID NO: 9) |

³The CDRs are determined according to IMGT.

TABLE F
OX40 VL CDR Amino Acid Sequences⁴

| VL CDR1 | VL CDR2 | VL CDR3 |
|---|---|---|
| (SEQ ID NO:) | (SEQ ID NO:) | (SEQ ID NO:) |
| NIGSKS | DDS | QVWDSSSDHVV |
| (SEQ ID NO: 10) | (SEQ ID NO: 11) | (SEQ ID NO: 12) |

⁴The CDRs are determined according to IMGT.

A 4-1BB×OX40 bispecific antibody or antigen binding fragment thereof that is bivalent for OX40 can comprise two OX40 binding domains, each comprising the six CDRs listed in Tables E and F above.

In some aspects of the 4-1BB×OX40 bispecific antibody or antigen binding fragment thereof described herein, a OX40 binding domain comprises the VH sequence provided in Table G

TABLE G
OX40 Variable Heavy Chain (VH)
Amino Acid Sequence

| SEQ ID NO | VH Amino Acid Sequence |
|---|---|
| 17 | QVQLVESGGGVVQPGRSLRLSCAASGFTLSYYGMHWVR |
| | QAPGKGLEWVAAISHDGSDKYYADSVKGRFTISRDNSK |
| | NRLYLQMNSLRAEDTAVYYCSNDQFDPWGQGTLVTVSS |

An OX40 binding domain described herein can comprise a VH comprising the CDRs of a VH sequence in Table G, e.g., the IMGT-defined CDRs, the Kabat-defined CDRs, the Chothia-defined CDRs.

In some aspects of the 4-1BB×OX40 bispecific antibody or antigen binding fragment thereof described herein, a OX40 binding domain comprises the VL sequence provided in Table H.

TABLE H
OX40 Variable Light Chain (VL)
Amino Acid Sequence

| SEQ ID NO. | VL Amino Acid Sequence |
|---|---|
| 18 | SYVLTQPPSVSVAPGKTARITCGGNNIGSKSVNWFQQK |
| | PGQAPVLVVYDDSGRPSGIPERFSGSTSGNTATLTISR |
| | VEAGDEADYYCQVWDSSSDHVVFGGGTKLTVL |

An OX40 binding domain described herein can comprise a VL comprising the CDRs of a VL sequence in Table I, e.g., the IMGT-defined CDRs, the Kabat-defined CDRs, the Chothia-defined CDRs, or the AbM-defined CDRs.

In some aspects, the 4-1BB×OX40 bispecific antibody or antigen binding fragment thereof described herein can comprise two OX40 binding domains, each comprising a VH provided in Table G and a VL provided in Table H. The VH provided in Table G and a VL provided in Table H can be on different polypeptides or can be on the same polypeptide. When the VH and VL are on the same polypeptide, they can be in either orientation (i.e., VH-VL or VL-VH), and they can be connected by a linker (e.g., a glycine-serine linker). In some aspects, the VH and VL are connected a glycine-serine linker that is at least 15 amino acids in length (e.g., 15-50 amino acids 15-40 amino acids, 15-30 amino acids, 15-25 amino acids or 15-20 amino acids). In some aspects, the VH and VL are connected a glycine-serine linker that is at least 20 amino acids in length (e.g., 20-50 amino acids 20-40 amino acids, 20-30 amino acids, or 20-25 amino acids).

In some aspects of the 4-1BB×OX40 bispecific antibody or antigen binding fragment described herein, an OX40 binding domain can comprise a VH comprising the CDRs of a VH sequence in Table G, e.g., the IMGT-defined CDRs, the Kabat-defined CDRs, the Chothia-defined CDRs, or the AbM-defined CDRs and a VL comprising the CDRs of a VL sequence in Table H, e.g., the IMGT-defined CDRs, the Kabat-defined CDRs, the Chothia-defined CDRs, or the AbM-defined CDRs.

C. 4-1BB and/or OX40 Binding Domains

In a 4-1BB or OX40 binding domain, the VH CDRs or VH and the VL CDRs or VL can be separate polypeptides or can be on the same polypeptide. When the VH CDRs or VH and the VL CDRs or VL are on the same polypeptide, they can be in either orientation (i.e., VH-VL or VL-VH).

When the VH CDRs or VH and the VL CDRs or VL are on the same polypeptide, they can be connected by a linker (e.g., a glycine-serine linker). The VH can be positioned N-terminally to a linker sequence, and the VL can be positioned C-terminally to the linker sequence. Alternatively, the VL can be positioned N-terminally to a linker sequence, and the VH can be positioned C-terminally to the linker sequence.

The use of peptide linkers for joining VH and VL regions is well-known in the art, and a large number of publications exist within this particular field. In some aspects, a peptide linker is a 15mer consisting of three repeats of a Gly-Gly-Gly-Gly-Ser amino acid sequence ((Gly₄Ser)₃) (SEQ ID NO:20). In some aspects, a suitable linker can be obtained by optimizing a simple linker (e.g., (Gly4Ser)n), wherein n=1-5 (SEQ ID NO:21) through random mutagenesis.

The 4-1BB and/or OX40 binding domain can be a humanized binding domain. The 4-1BB and/or OX40 binding domain can be a rat binding domain. The 4-1BB and/or OX40 binding domain can be a murine binding domain. In some aspects, a 4-1BB×OX40 bispecific antibody comprises a humanized 4-1BB binding domain and a rat OX40 binding domain. In some aspects, a 4-1BB×OX40 bispecific antibody comprises a humanized 4-1BB binding domain and a murine OX40 binding domain. In some aspects, a 4-1BB×OX40 bispecific antibody comprises a humanized 4-1BB binding domain and a humanized OX40 binding domain.

In some aspects of the 4-1BB×OX40 bispecific antibody or antigen binding fragment thereof described herein, the 4-1BB and/or OX40 binding domain can be an scFv. In some aspects, all of the 4-1BB and OX40 binding domains in a 4-1BB×OX40 bispecific antibody or antigen binding fragment thereof are scFvs. In some aspects, at least one 4-1BB or OX40 binding domain in a 4-1BB×OX40 bispecific antibody or antigen binding fragment thereof is an scFv. In some aspects of the 4-1BB×OX40 bispecific antibody or antigen binding fragment thereof described herein, a polypeptide comprises a 4-1BB binding domain (e.g., an scFv) and an OX40 binding domain (e.g., an scFv).

In some aspects of the 4-1BB×OX40 bispecific antibody or antigen binding fragment described herein, the 4-1BB scFv comprises the amino acid sequence of SEQ ID NO: 16. In some aspects of the 4-1BB×OX40 bispecific antibody or antigen binding fragment described herein, the OX40 scFv comprises the amino acid sequence of SEQ ID NO: 19.

In some aspects, the 4-1BB and/or OX40 binding domain can comprise a VH and a VL on separate polypeptide chains. In some aspects, all of the 4-1BB and OX40 binding domains in a 4-1BB×OX40 bispecific antibody or antigen binding fragment thereof comprise a VH and a VL on separate polypeptide chains. In some aspects, the 4-1BB and/or OX40 binding domain can comprise a VH and a VL on the same polypeptide chain. In some aspects, all of the 4-1BB or OX40 binding domains in a 4-1BB×OX40 bispecific antibody or antigen binding fragment thereof comprises a VH and a VL on the same polypeptide chains.

In some aspects of the 4-1BB×OX40 bispecific antibody or antigen binding fragment thereof described herein the 4-1BB×OX40 bispecific antibody or antigen binding fragment thereof comprises two polypeptides, each polypeptide comprising, in order from amino-terminus to carboxyl-terminus, a first antigen-binding domain, a linker (e.g., wherein the linker is a hinge region), an immunoglobulin constant region, and a second antigen-binding domain. This configuration is also referred to herein as an ADAPTIR™ format. An exemplary molecule is referred to throughout the present disclosure as FXX01102.

In such a format, the 4-1BB×OX40 bispecific antibody or antigen binding fragment thereof comprises a dimer of two polypeptides, each polypeptide comprising in order from amino-terminus to carboxyl-terminus, a first scFv, a hinge region, an immunoglobulin constant region, and a second scFv, wherein (a) the first scFv comprises a human 4-1BB antigen-binding domain, and the second scFv comprises a human OX40 antigen-binding domain or (b) the first scFv comprises a human OX40 antigen-binding domain and the second scFv comprises a human 4-1BB antigen-binding domain.

As provided herein, an antibody or antigen binding fragment thereof or polypeptide comprising any of the CDR, VH, VL, scFv, and/or hinge provided herein may further comprise an immunoglobulin constant region. The presence of the constant region extends the half-life of the bispecific antibody as compared to a similar bispecific antibody without a constant region. An immunoglobulin constant region can be located, for example between a hinge and a 4-1BB binding domain (e.g., a 4-1BB binding scFv). An immunoglobulin constant region can also be located between a hinge and an OX40-binding domain (e.g., an OX-40 binding scFv). In some aspects, a polypeptide comprises, in order from amino-terminus to carboxyl-terminus, a hinge region, an immunoglobulin constant region, and an antigen-binding domain (e.g., an scFv).

In some aspects, the immunoglobulin constant region comprises immunoglobulin CH2 and CH3 domains of IgG1, IgG2, IgG3, IgG4, IgA1, IgA2 or IgD, optionally wherein the IgG is human. In some cases, the immunoglobulin constant region comprises immunoglobulin CH2 and CH3 domains of IgG1 (e.g., human IgG1). In some aspects, the polypeptide does not contain a CH1 domain.

In some aspects, the 4-1BB×OX40 bispecific antibody or antigen binding fragment thereof described herein comprises the 4-1BB VH CDR1, CDR2, and CDR3 sequences of SEQ ID NOs:1-3, respectively, the 4-1BB VL CDR1, CDR2, and CDR3 sequences of SEQ ID NOs:4-6, respectively, the OX40 VH CDR1, CDR2, and CDR3 sequences of SEQ ID NOs:7-9, respectively, and the OX40 VL CDR1, CDR2, and CDR3 sequences of SEQ ID NOs:10-12, respectively.

In some aspects, the human 4-1BB binding domain and the human OX40 binding domain are on the same polypeptide. In some aspects, the human 4-1BB binding domain is N-terminal to the human OX40 binding domain. In some aspects, the human 4-1BB binding domain is C-terminal to the human OX40 binding domain. In some aspects, the immunoglobulin constant region comprises immunoglobulin CH2 and CH3 domains of IgG1.

In some aspects, the antibody does not contain a CH1 domain.

In some aspects, the immunoglobulin constant region comprises a human IgG1 CH2 domain comprising the substitutions E233P, L234A, L235A, G237A, and K322A and a deletion of G236 according to the EU numbering system. In some aspects, the antibody or antigen-binding fragment thereof comprises a linker between an immunoglobulin constant region and the human 4-1BB binding domain and/or between an immunoglobulin constant region and the human OX40 binding domain. In some aspects, the linker between the immunoglobulin constant region and the human 4-1BB binding domain and/or between the immunoglobulin constant region and the human OX40 binding domain comprises 10-30 amino acids, 15-30 amino acids, or 20-30 amino acids. In some aspects, the linker between the immunoglobulin constant region and the human 4-1BB binding domain or between the immunoglobulin constant region and the human OX40 binding domain comprises the amino acid sequence (Gly4Ser)n, wherein n=1-5 (SEQ ID NO:21), optionally wherein n=1.

D. Compositions Comprising 4-1BB and OX40 Binding Domains

In some aspects, the pharmaceutical composition described herein comprises a 4-1BB×OX40 bispecific antibody or antigen binding fragment thereof comprising a dimer of two polypeptides, each polypeptide comprising in order from amino-terminus to carboxyl-terminus, a first scFv, a hinge region, an immunoglobulin constant region, and a second scFv, wherein (a) the first scFv comprises a human 4-1BB antigen-binding domain, and the second scFv comprises a human OX40 antigen-binding domain or (b) the first scFv comprises a human OX40 antigen-binding domain and the second scFv comprises a human 4-1BB antigen-binding domain. In some aspects the dimer is a homodimer.

In some aspects, the 4-1BB×OX40 bispecific antibody or antigen binding fragment thereof described herein comprises the amino acid sequence of SEQ ID NO:13.

Some aspects of the present disclosure provide a pharmaceutical composition comprising a 4-1BB×OX40 bispecific antibody or antigen binding fragment thereof comprising a human 4-1BB antigen-binding domain and a human OX40 antigen-binding domain, wherein: (a) the 4-1BB antigen-binding domain comprises a (i) a VH-CDR 1 comprising the amino acid sequence of SEQ ID NO:1; (ii) a VH-CDR2 comprising the amino acid sequence of SEQ ID NO:2; (iii) a VH-CDR3 comprising the amino acid sequence of SEQ ID NO:3; (iv) a light chain variable domain (VL)-CDR1 comprising the amino acid sequence of SEQ ID NO:4; (v) a VL-CDR2 comprising the amino acid sequence of SEQ ID NO:5; and (vi) a VL-CDR3 comprising the amino acid sequence of SEQ ID NO:6; and (b) the OX40 antigen-binding domain comprises a (i) a VH-CDR1 comprising the amino acid sequence of SEQ ID NO:7; (ii) a VH-CDR2 comprising the amino acid sequence of SEQ ID NO:8; (iii) a VH-CDR3 comprising the amino acid sequence of SEQ ID NO:9; (iv) a VL-CDR1 comprising the amino acid sequence of SEQ ID NO:10; (v) a VL-CDR2 comprising the amino acid sequence of SEQ ID NO:11; and (vi) a VL-CDR3 comprising the amino acid sequence of SEQ ID NO: 12.

Some aspects of the present disclosure provide a pharmaceutical composition comprising a 4-1BB×OX40 bispecific antibody or antigen binding fragment thereof comprising a human 4-1BB antigen-binding domain and a human OX40 antigen-binding domain wherein: (a) the 4-1BB antigen-binding domain comprises a heavy chain variable region comprising the amino acid sequence of SEQ ID NO: 14 and a light chain variable region comprising the amino acid sequence of SEQ ID NO: 15; and (b) the OX40 antigen-binding domain comprises a heavy chain variable region comprising the amino acid sequence of SEQ ID NO: 17 and a light chain variable region comprising the amino acid sequence of SEQ ID NO: 18.

Some aspects of the present disclosure provide a pharmaceutical composition comprising a 4-1BB×OX40 bispecific antibody or antigen binding fragment thereof comprising a human 4-1BB antigen-binding domain and a human OX40 antigen-binding domain, wherein: (a) the 4-1BB antigen-binding domain comprises a scFv comprising the amino acid sequence of SEQ ID NO: 16; and (b) the OX40 antigen-binding domain comprises a scFv comprising the amino acid sequence of SEQ ID NO: 19.

Some aspects of the present disclosure provide a pharmaceutical composition comprising a 4-1BB×OX40 bispecific antibody or antigen binding fragment thereof comprising a human 4-1BB antigen-binding domain and a human OX40 antigen-binding domain comprising the amino acid sequence of SEQ ID NO: 13.

Some aspects of the present disclosure provide a pharmaceutical composition comprising: (a) 10-50 mg/mL of a 4-1BB×OX40 bispecific antibody or antigen-binding fragment thereof, wherein the antibody or antigen-binding fragment thereof comprises: (i) a 4-1BB antigen-binding domain comprising a VH-CDR 1 comprising the amino acid sequence of SEQ ID NO:1; a VH-CDR2 comprising the amino acid sequence of SEQ ID NO:2; a VH-CDR3 comprising the amino acid sequence of SEQ ID NO:3; a light chain variable domain (VL)-CDR1 comprising the amino acid sequence of SEQ ID NO:4; a VL-CDR2 comprising the amino acid sequence of SEQ ID NO:5; and a VL-CDR3 comprising the amino acid sequence of SEQ ID NO:6; and an OX40 antigen-binding domain comprising a VH-CDR1 comprising the amino acid sequence of SEQ ID NO:7; a VH-CDR2 comprising the amino acid sequence of SEQ ID NO:8; a VH-CDR3 comprising the amino acid sequence of SEQ ID NO:9; a VL-CDR1 comprising the amino acid sequence of SEQ ID NO:10; a VL-CDR2 comprising the amino acid sequence of SEQ ID NO:11; and a VL-CDR3 comprising the amino acid sequence of SEQ ID NO:12; (ii) a 4-1BB antigen-binding domain comprising a heavy chain variable region comprising the amino acid sequence of SEQ ID NO: 14 and a light chain variable region comprising the amino acid sequence of SEQ ID NO: 15; and a OX40 antigen-binding domain comprising a heavy chain variable region comprising the amino acid sequence of SEQ ID NO: 17 and a light chain variable region comprising the amino acid sequence of SEQ ID NO: 18; (iii) a 4-1BB antigen-binding domain comprising a scFv comprising the amino acid sequence of SEQ ID NO: 16; and an OX40 antigen-binding domain comprising a scFv comprising the amino acid sequence of SEQ ID NO: 19; or (iv) the amino acid sequence of SEQ ID NO: 13; (b) about 5 mM to about 15 mM glutamate; (c) about 25 mM to about 50 mM leucine; (d) about 2% to about 8% w/v sucrose; and (e) about 0.01% to about 0.03% polysorbate-80; wherein the pH of the composition is from about 4.3 to about 4.7.

Some aspects of the present disclosure provide a pharmaceutical composition comprising: (a) 10 mg/mL of a 4-1BB×OX40 bispecific antibody or antigen-binding fragment thereof; wherein the antibody or antigen-binding fragment thereof comprises: (i) a 4-1BB antigen-binding domain comprising a VH-CDR 1 comprising the amino acid sequence of SEQ ID NO:1; a VH-CDR2 comprising the amino acid sequence of SEQ ID NO:2; a VH-CDR3 comprising the amino acid sequence of SEQ ID NO:3; a light chain variable domain (VL)-CDR1 comprising the amino acid sequence of SEQ ID NO:4; a VL-CDR2 comprising the amino acid sequence of SEQ ID NO:5; and a VL-CDR3 comprising the amino acid sequence of SEQ ID NO:7; and (ii) an OX40 antigen-binding domain comprising a VH-CDR1 comprising the amino acid sequence of SEQ ID NO:7; a VH-CDR2 comprising the amino acid sequence of SEQ ID NO:8; a VH-CDR3 comprising the amino acid sequence of SEQ ID NO:9; a VL-CDR1 comprising the amino acid sequence of SEQ ID NO:10; a VL-CDR2 comprising the amino acid sequence of SEQ ID NO:11; and a VL-CDR3 comprising the amino acid sequence of SEQ ID NO:12; (b) 10 mM glutamate; (c) 40 mM leucine; (d) 3% w/v sucrose; and (e) 0.02% polysorbate-80; wherein the pH of the composition is 4.5.

In some aspects, the pharmaceutical composition comprises (a) the 4-1BB antigen-binding domain comprising a heavy chain variable region comprising the amino acid sequence of SEQ ID NO: 14 and a light chain variable region comprising the amino acid sequence of SEQ ID NO: 15; and (b) the OX40 antigen-binding domain comprises a heavy chain variable region comprising the amino acid sequence of SEQ ID NO: 17 and a light chain variable region comprising the amino acid sequence of SEQ ID NO: 18.

### IV. METHODS AND USES

The present disclosure provides pharmaceutical compositions comprising antibodies or antigen binding fragments that bind to 4-1BB and/or OX40, which compositions are useful in a variety of applications including, but not limited to, therapeutic treatment methods, such as the treatment of cancer. In some aspects, the pharmaceutical compositions are useful for inhibiting tumor growth and/or reducing tumor volume. The methods of use can be in vitro or in vivo methods. The pharmaceutical compositions described herein can include the use of any of the disclosed antibodies or antigen binding fragments for use in therapy.

The present disclosure provides methods of treating cancer in a subject comprising administering to the subject an effective amount of a pharmaceutical compositions comprising (a) a bispecific antibody or antigen-binding fragment thereof that specifically binds 4-1BB and OX40; (b) about 5 mM to about 15 mM of a stability promoting buffer; (c) about 25 mM to about 50 mM of a stabilizing amino acid; (d) about 2% to about 8% w/v of a sugar; and (e) about 0.01% to about 0.03% of a surfactant.

In some aspects, the cancer is a cancer including, but are not limited to, melanoma, kidney cancer, pancreatic cancer, lung cancer, colon cancer/intestinal cancer, stomach cancer, prostate cancer, ovarian cancer, breast cancer, liver cancer, brain cancer, and hematological cancers. The cancer may be a primary tumor or may be advanced or metastatic cancer. In some aspects, the cancer is a solid tumor. For instance, the present disclosure includes use of the bispecific antibodies for treatment of sarcoma, carcinoma, and lymphoma. The disclosure provides methods for treating a human subject with a sarcoma, carcinoma, or lymphoma by administering to the subject a therapeutically effective amount of a pharmaceutical composition described herein.

In some aspects, the disclosure provides methods of treating a human subject with a tumor or cancerous tissue that contains tumor infiltrating lymphocytes. The disclosure provides treating a human subject with a tumor containing lymphocytes that express 4-1BB and OX40. In some aspects, the disclosure provides administering to a human subject with a solid tumor a therapeutically effective amount of a pharmaceutical composition comprising (a) a bispecific antibody or antigen-binding fragment thereof that specifically binds 4-1BB and OX40; (b) about 5 mM to about 15 mM of a stability promoting buffer; (c) about 25 mM to about 50 mM of a stabilizing amino acid; (d) about 2% to about 8% w/v of a sugar; and (e) about 0.01% to about 0.03% of a surfactant.

Some aspects of the present disclosure provide methods of enhancing an immune response in a subject comprising administering a therapeutically effective amount of a pharmaceutical composition comprising a 4-1BB×OX40 bispecific antibody or antigen binding fragment thereof, as disclosed herein, to the subject.

Some aspects of the present disclosure provide methods of agonizing a T cell co stimulatory pathway in a subject comprising administering a pharmaceutical composition comprising a 4-1BB×OX40 bispecific antibody or antigen binding fragment thereof, as disclosed herein, to the subject.

Some aspects of the present disclosure provide methods of increasing the proliferation of NK cells and/or T cells (e.g., CD4+ T cells and/or CD8+ T cells) in a subject comprising administering a therapeutically effective amount a pharmaceutical composition comprising a 4-1BB×OX40 bispecific antibody or antigen binding fragment thereof, as disclosed herein, to the subject.

Some aspects of the present disclosure provide methods of increasing the number of tumor infiltrating lymphocytes in a subject by administering to the subject a therapeutically effective amount of a pharmaceutical composition comprising a 4-1BB×OX40 bispecific antibody or antigen binding fragment thereof, as disclosed herein, to the subject.

Some aspects of the present disclosure provide methods of increasing the expression of granzymes by tumor infiltrating lymphocytes in a subject by administering to the subject a therapeutically effective amount of a pharmaceutical composition comprising a 4-1BB×OX40 bispecific antibody or antigen binding fragment thereof, as disclosed herein, to the subject.

In some aspects, the subject is a human.

In some aspects, provided herein are pharmaceutical compositions comprising 4-1BB×OX40 bispecific antibodies or antigen binding fragments thereof, for use as a medicament. In some aspects, provided herein are pharmaceutical compositions comprising a 4-1BB×OX40 bispecific antibodies or antigen binding fragments thereof for use in a method for the treatment of cancer

### V. KITS AND CONTAINERS

The present disclosure also provides containers comprising any pharmaceutical formulation described and exemplified herein.

In some aspects, the present disclosure provides kits comprising any of the pharmaceutical formulations and/or bispecific antibodies or antigen binding fragments thereof described and exemplified herein. The kits can be used to supply pharmaceutical formulations, antibodies, antigen-binding fragments thereof, and other agents for use in diagnostic, basic research, or therapeutic methods, among others. In some aspects, the kits comprise any one or more of the pharmaceutical formulations, antibodies or antigen-binding fragments thereof, and/or bispecific antibodies described and exemplified herein, and instructions for using the one or more pharmaceutical formulations, antibodies, or antigen-binding fragments thereof in a method of treating cancer in a subject. In some aspects, the kits comprise any one or more of the pharmaceutical formulations, antibodies or antigen-binding fragments thereof, and/or bispecific antibodies described and exemplified herein and instructions for using the one or more pharmaceutical formulations, antibodies, or antigen-binding fragments thereof for use in the treatment of a solid tumor cancer.

### EXAMPLES

It is understood that the examples described herein are for illustrative purposes only and that various modifications or changes in light thereof will be suggested to persons skilled in the art and are to be included within the spirit and purview of this application.

### Example 1. Use of Different Excipients to Stabilize a Protein to Minimize Aggregation and or Degradation

Different components and excipients may bind, interact and/or stabilize different regions of a protein based on their chemical characteristics. Accordingly, it is useful to screen a variety of different types of excipients to determine their impact on the protein formulation, such as the subset listed in Table 1 below.

The impact of different formulations can be measured in a variety of means. One approach is to store the formulated protein at different temperatures, including both potential storage conditions for the drug after it has been manufactured (“intended storage conditions”) as well as accelerated storage conditions (temperatures higher than the intended storage conditions). When performing a storage stability experiment, samples are removed at different timepoints and analyzed by different techniques, such as analytical Size Exclusion High Performance Liquid Chromatography (SE-HPLC). Measuring the change in purity over time using SE-HPLC can be useful in assessing whether different formulations inhibit or promote aggregation or degradation. Analytical Cation Exchange Chromatography (CEX-HPLC) and/or capillary isoelectric focusing (cIEF) can be used to monitor the change in the charge variant profile of the protein to determine whether different formulation conditions are inhibiting or promoting charge-related changes in the protein structure.

In addition to evaluating different formulations using storage stability experiments, other methods can be employed to characterize the biophysical impact on protein stability. For example, determining the melting point (T_{M}, mid-point of the thermal melt transition) using Differential Scanning Fluorimetry (DSF) or Differential Scanning Calorimetry (DSC) can be helpful in assessing whether different formulations are stabilizing (increasing T_{M}) or detabilizing (decreasing T_{M}) the thermal stability of a protein. Another biophysical method that can be used to assess the stabilizing effect of a formulation is the determination of B₂₂, the particle interaction parameter or second virial coefficient. Using light scattering measurements at different protein concentrations in a given formulation, the slope of the line can be used to determine whether the solution is beneficial (increasing, positive slope) or disadvantageous (decreasing, negative slope). Dynamic light scattering (DLS) monitoring at 266 nm can also be used to determine the temperature of aggregation (T_{agg}), which is also useful in comparing different formulations.

TABLE 1
Examples of components that could be
included in a protein formulation

| Component | Classification |
|---|---|
| Leucine | Amino Acid- hydrophobic |
| Glutamic Acid | Amino Acid - negatively charged |
| Glycine | Amino Acid neutral |
| Arginine | Amino Acid - positively charged |
| Histidine | Amino Acid - positively charged |
| Lysine | Amino Acid - positively charged |
| Methionine | Amino Acid - anti-oxidant |
| Ascorbic Acid | Anti-oxidant |
| Glycerol | Bulking agent |
| EDTA | Chelator |
| Sodium Chloride | Salt |
| Calcium Chloride | Salt |
| Magnesium Chloride | Salt |
| Potassium Chloride | Salt |
| Mannitol | Sugar |
| Sorbitol | Sugar |
| Sucrose | Sugar |
| Polysorbate-80 | Detergent |

### Example 2. Evaluation of Different Buffers, pH, and Sucrose Concentration

The FXX01102 bispecific protein was expressed by CHO cells in bioreactors and purified to near-homogeneity (>95% pure by SE-HPLC) using column chromatography. It was concentrated, buffer-exchanged, and formulated into a series of different compositions shown in Table 2 below. All buffers were utilized at 10 mM and the FXX01102 protein concentration was 10 mg/mL in each solution. The purpose of this study was to determine the effect of different buffers, pH, and sucrose concentrations on the stability of the FXX01102 bispecific antibody. The onset of aggregation as a function of temperature (T_{agg}) and Tₘ (mid-point of the melting curve) was determined using the Uncle instrument (Unchained Labs). T_{agg} was measured via light scattering measurements at 266 nm as function of increasing temperature.

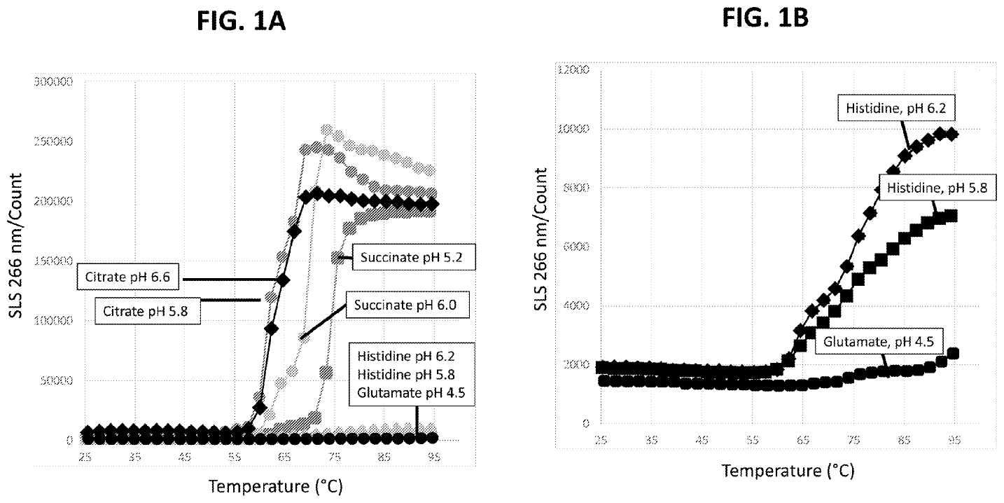

Comparison of the Tₘ values across the sample set suggested that pH, buffer, and sucrose concentration did not have a large impact on the unfolding of the first domain (Tₘ₁), as the values all fell within ˜61-62° C. (data not shown). However, comparison of the Dynamic Light Scattering (DLS) data at 266 nm showed differences in the formation of insoluble particulate in the samples (FIGS. 1A and 1B). The sucrose concentration caused minor changes relative to the impact of buffer and pH, so only the 2% w/v sucrose data was plotted for succinate, histidine, and citrate, and the 8% w/v glutamate buffer. As shown in FIG. 1A, the two citrate buffers at pH 5.8 and 6.6 had very similar T_{agg} curves, with the pH 6.6 trace shifted to slightly higher temperatures, reflecting a more stable solution condition for FXX01102. The T_{agg} data for citrate suggested it was the least stability-promoting buffer among those tested, followed by succinate. The low pH succinate formulation at pH 5.2 provided a higher T_{agg} value than the pH 6.0 solution. The histidine and glutamate buffers all showed minimal increase in light scatter and were identified as the preferred formulations in the set. A plot of the His and Glu buffers (FIG. 1B) showed that the glutamate had the lowest light scattering signal among those evaluated. The histidine formulations showed the same trend as the succinate formulations, with the lower pH 5.8 formulation performing better than the higher pH 6.2 solution. Because similar pH values were used with different buffering agents, the differences in the T_{agg} were not a result of pH alone, but likely a combination of pH and charge characteristics of the buffer. Based in part on these results, glutamate became the primary buffering agent in subsequent experiments

TABLE 2
Buffers evaluated for impact on Tₘ and T_{agg}

| Buffer | Sucrose | Polysorbate-80 | |
|---|---|---|---|
| (10 mM) | (w/v) | (w/v) | pH |
| Succinate | 2% | 0.02 | 5.2 |
| Succinate | 6% | 0.02 | 5.2 |
| Succinate | 2% | 0.02 | 6.0 |
| Succinate | 6% | 0.02 | 6.0 |
| Histidine | 2% | 0.02 | 5.8 |
| Histidine | 6% | 0.02 | 5.8 |
| Histidine | 2% | 0.02 | 6.2 |
| Histidine | 6% | 0.02 | 6.2 |
| Citrate | 2% | 0.02 | 5.8 |
| Citrate | 6% | 0.02 | 5.8 |
| Citrate | 2% | 0.02 | 6.6 |
| Citrate | 6% | 0.02 | 6.6 |
| Glutamate | 8% | 0.02 | 4.5 |

### Example 3. Evaluation of the Impact of NaCl on a Low pH Formulation

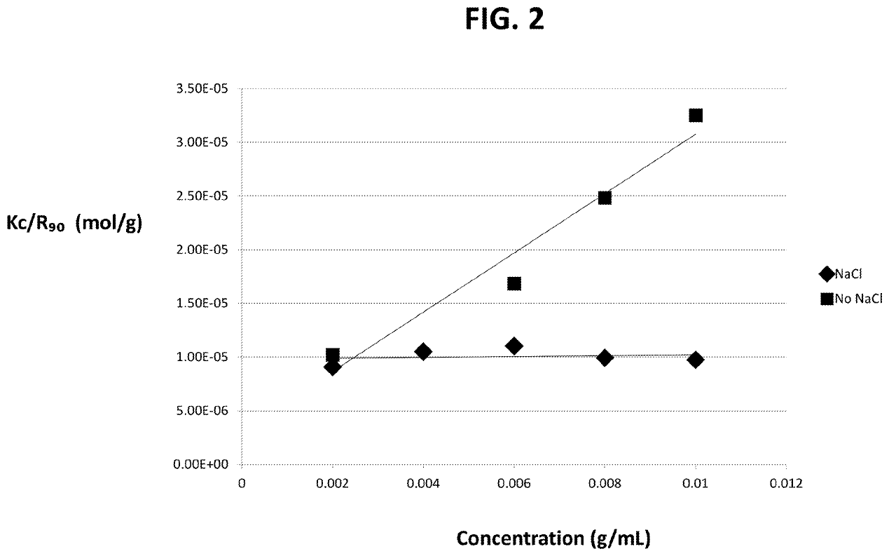

The FXX01102 bispecific protein was concentrated and exchanged into a solution of 10 mM glutamate, 8% weight/volume sucrose, and 0.02% weight/volume polysorbate-80 at pH 4.5, with a final protein concentration of 8 mg/mL, with or without the inclusion of 100 mM sodium chloride (NaCl). A dilution series was generated at protein concentrations of 0, 2, 4, 6, and 8 mg/mL. These solutions were analyzed on the “Uncle” instrument, manufactured by Unchained Laboratories. The goal was to determine whether the inclusion of sodium chloride improved the stabilizing properties of the glutamate/sucrose/Polysorbate-80 formulation. The B₂₂ was examined for these two solutions, and it was evident that the addition of NaCl to the formulation caused a significant decrease in the slope, equating to a destabilizing effect and promoting protein-protein interactions that could lead to aggregation (FIG. 2). Sodium chloride was therefore not evaluated further in these studies.

### Example 4. Evaluation of the Impact of Arginine, Methionine, Leucine and Glycine on the B22 Value of FXX01102 Formulations

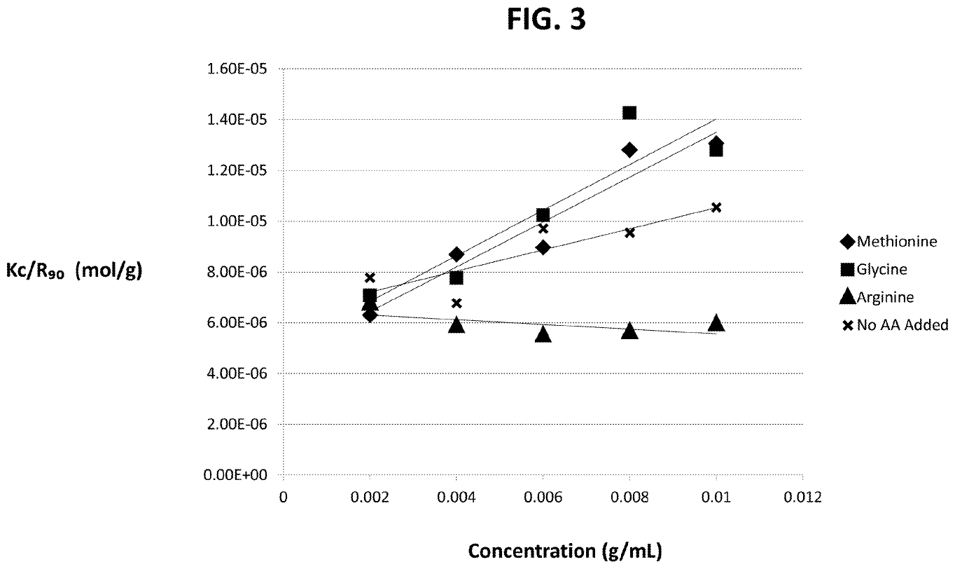

The amino acids methionine, arginine, and glycine were also investigated for the stabilizing properties they might exert on FXX01102. A 10 mg/mL solution of FXX01102 was prepared in 10 mM glutamate, 4% w/v sucrose, at pH 4.5, with no additional excipients, with either 100 mM arginine, 50 mM methionine or 50 mM glycine. A dilution series at 0, 2, 4, 6, 8, and 10 mg/mL was analyzed on the Uncle to examine the impact on B₂₂. A comparison of the B₂₂ slopes (FIG. 3) shows that both glycine and methionine appear to increase the B₂₂ slope value compared to arginine, which decreases the slope compared to the formulation in which no additional amino acid was added. This suggested that non-charged or more hydrophobic amino acids may have a more beneficial effect on FXX01102 stability compared to adding salts or other charged amino acids.

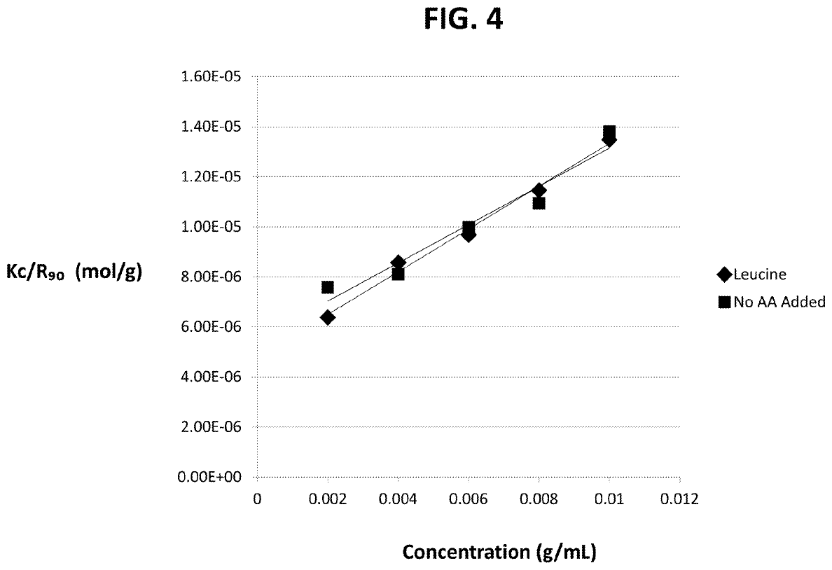

In order to further explore the impact of hydrophobic amino acids on FXX01102 stability, B₂₂ slopes of formulations with or without 25 mM leucine were compared. A dilution series of FXX01102 in 10 mM glutamate, 4% w/v sucrose, at pH 4.5, with or without leucine was examined. The slope of the leucine-containing formulation was slightly higher than that of the protein solution without leucine, suggesting that leucine may exert a stabilizing effect on the protein and reduce self-interaction properties (FIG. 4).

### Example 5. Evaluation of the Impact of Magnesium Sulfate, Lysine, and Leucine on the Aggregation Temperature (T_{agg}) of FXX01102 Formulations

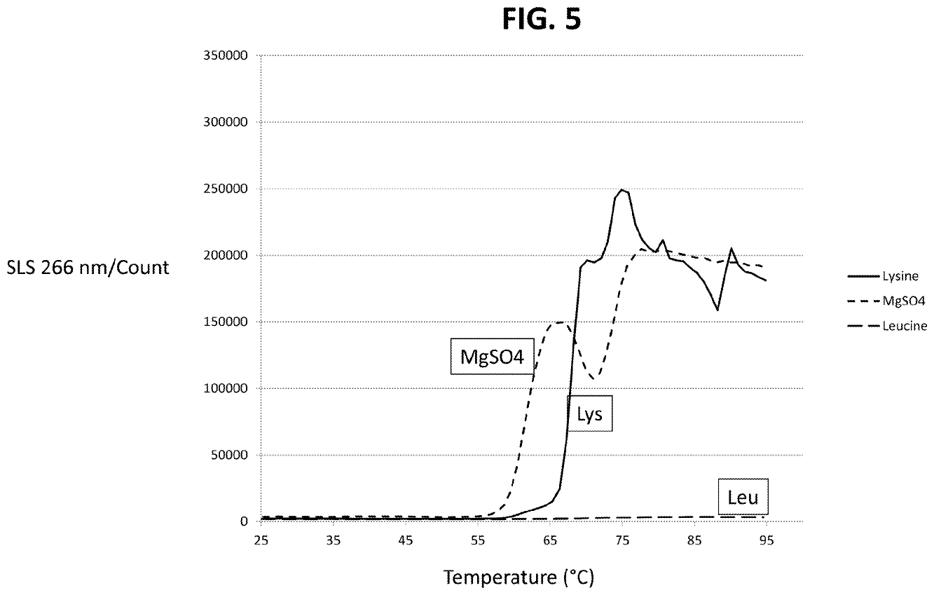

For a subset of formulations, the onset of aggregation as a function of temperature (T_{agg}) was determined using the Uncle instrument (Unchained Labs). T_{agg} was measured via light scattering measurements at 266 nm as function of increasing temperature. FXX01102 in 10 mM glutamate, 4% w/v sucrose, at pH 4.5, with 25 mM leucine, MgSO₄ and lysine. A comparison of the plots of scatter at 266 nm versus temperature showed that both MgSO₄ and lysine caused an increase in scatter at much lower temperatures compared to the formulation with no additional excipients (FIG. 5). The sample containing leucine did not show any increase in scatter over the full range of temperature that was evaluated. This indicated that the improvement in B₂₂ obtained with leucine (described in the previous example) may have translated into an increased resistance to aggregation when FXX01102 was formulated.

### Example 6. Stability Evaluation of FXX01102 Formulations at 20° C.

An experiment was performed to evaluate the stability of FXX01102 when stored at 20° C. All formulations shown in Table 3 below contained 10 mM glutamate as the buffering agent and 0.02% Polysorbate. In addition, all formulations contained a sugar component, either 3 or 8% w/v sucrose, 3% trehalose, or 3% sorbitol. Based on previous data, whether the addition of Leu, Met or Gly amino acids improved the stability of FXX01102 was investigated. A formulation with improved stability should show less aggregate formation as measured by size exclusion ultra pressure liquid chromatography (SE-UPLC).

FXX01102 was purified from clarified bioreactor supernatant using two chromatography columns to achieve a starting purity of >95%. This material was buffer-exchanged into glutamate using a UF/DF system and at least 10 diavolumes of buffer and concentrated to 10 mg/mL. After the different formulations were prepared, 200 μL aliquots in 0.5 mL Sarstedt vials were made. The pH was determined at the start of the study. Most samples had a pH of ˜4.6. The initial % High Molecular Weight content (% HMW) using SE-UPLC was determined. Vials were then placed in a 20° C. and measured at 3 weeks, 6 weeks, 3, 6, and 7 months (for a subset of formulations) after storage. At the appropriate timeframe, vials were removed from the freezer and analyzed. A larger 3.75 mL aliquot was prepared and taken through three freeze/thaw (3×F/T) cycles. This served as an additional stress-test to compare the different formulations. This data is summarized in Table 3 below. After evaluation of the 3 month data, only a subset of samples was analyzed at 6 and 7 months to focus in on particular formulations of interest.

Comparison of the sorbitol and trehalose-containing formulations (ID #5, 6) to the other formulations in set indicated that they contribute to aggregate formation. These two formulations were the worst-performing conditions analyzed, based on the % HMW of the 3×F/T samples, the 3 month, and 6 month timepoints. Therefore, sucrose was identified as the preferred sugar component. The aggregate data for 3% and 8% sucrose (ID #2 and #3, respectively) indicated that higher concentrations of sucrose did not have a significant benefit for long-term storage, as the % HMW at 3 and 6 months for these formulations were quite similar. However, it was observed that there was less aggregate present after 3×F/T with the higher sucrose concentration (6.3 vs. 8.8%).

Based on the B₂₂ analysis performed above, it was predicted that Met, Gly and Leu would have a positive impact on the storage stability of FXX01102. Three concentrations of each amino acid was analyzed for their impact on 20° C. stability. For each, increasing concentration led to less aggregate formation over time. For Met (ID #10, 11, 12), Gly (ID #13, 14, 15), and Leu (ID #7, 8, 9), there was a clear trend of lower % HMW over time as a function of the concentration of amino acid excipient added. Leucine appeared to have the greatest stabilizing impact, as formulation ID #9 had the lowest aggregate formation in both the 3×F/T analysis and over time. Formulations that included combinations of amino acids were also investigated (ID #16, 17, 18) and indicated that there was some benefit to this approach. However, comparison of the % HMW of the combination to formulations that just contained an equivalent concentration of Leu (i.e., #16 vs #8, or #18 vs. #8) suggested that Leu was the primary driver of this effect. The Tₘ and T_{agg} values for ID #7, 8 and 9 were determined in order to see whether increasing concentrations of leucine from 10 to 40 mM had a big impact on either of these two parameters. The differences were relatively small, with a Tₘ of 61° C. for all three buffers and no significant changes in the light scatter determined at 266 nm.

TABLE 3
Buffers evaluated for impact on % HMW after storage at −20 ° C.

| | | | Assay | | | | | | | |
|---|---|---|---|---|---|---|---|---|---|---|
| | | | pH | SE-UPLC, % HMW | | | | | | |
| Sample | | | Initial | | 3x | 3 | 6 | 3 | 6 | 7 |
| ID | Sugar | Excipient | pH | Initial | F/T | Week | Week | Month | Month | Month |
| 1 | 8% | N/A | 4.67 | 4.9 | 6.3 | 5.3 | 5.7 | 7.9 | 11.9 | NT |
| | Sucrose | | | | | | | | | |
| 2 | 3% | N/A | 4.63 | 4.9 | 8.8 | 5.3 | 5.7 | 8 | 11.7 | 11.9 |
| | Sucrose | | | | | | | | | |
| 3 | 3% | N/A | 4.47 | 3.1 | 7.3 | 3.8 | 3.9 | 6.5 | 10.7 | NT |
| | Sucrose | | | | | | | | | |
| 4 | 3% | N/A | 4.75 | 6.5 | 9.7 | 6.8 | 7.3 | 9.1 | 12 | NT |
| | Sucrose | | | | | | | | | |
| 5 | 3% | N/A | 4.64 | 5 | 13.7 | 7.1 | 9.1 | 17.3 | NT | NT |
| | Trehalose | | | | | | | | | |
| 6 | 3% | N/A | 4.62 | 5 | 15.3 | 10.1 | 13.8 | 30.1 | NT | NT |
| | Sorbitol | | | | | | | | | |
| 7 | 3% | 10 mM | 4.63 | 4.8 | 7.2 | 4.9 | 5 | 5.7 | 7.5 | NT |
| | Sucrose | Leu | | | | | | | | |
| 8 | 3% | 25 mM | 4.62 | 4.6 | 6.2 | 4.6 | 4.7 | 5 | 5.2 | 5.1 |
| | Sucrose | Leu | | | | | | | | |
| 9 | 3% | 40 mM | 4.62 | 4.3 | 5.2 | 4 | 4.2 | 4.4 | 4.3 | 4.3 |
| | Sucrose | Leu | | | | | | | | |
| 10 | 3% | 10 mM | 4.61 | 4.9 | 8.1 | 6 | 6.3 | 8.3 | NT | NT |
| | Sucrose | Met | | | | | | | | |
| 11 | 3% | 25 mM | 4.62 | 4.7 | 6.8 | 5.2 | 5.1 | 6.5 | NT | 11.3 |
| | Sucrose | Met | | | | | | | | |
| 12 | 3% | 40 mM | 4.61 | 4.5 | 6.8 | 4.6 | 4.8 | 5.7 | NT | NT |
| | Sucrose | Met | | | | | | | | |
| 13 | 3% | 25 mM | 4.62 | 5.2 | 8.4 | 5.5 | 5.8 | 7.3 | NT | NT |
| | Sucrose | Gly | | | | | | | | |
| 14 | 3% | 50 mM | 4.62 | 5.5 | 8.3 | 5.6 | 5.8 | 7.1 | NT | 9.2 |
| | Sucrose | Gly | | | | | | | | |
| 15 | 3% | 75 mM | 4.64 | 5.6 | 7.8 | 5.8 | 5.9 | 6.6 | NT | NT |
| | Sucrose | Gly | | | | | | | | |
| 16 | 3% | 25 mM | 4.62 | 4.2 | 5.7 | 4.1 | 4.3 | 4.8 | NT | 5.3 |
| | Sucrose | Leu, 25 | | | | | | | | |
| | | mM Met | | | | | | | | |
| 17 | 3% | 25 mM | 4.68 | 4.6 | 5.8 | 5.1 | 5 | 6.1 | NT | NT |
| | Sucrose | Met, 50 | | | | | | | | |
| | | mM Gly | | | | | | | | |
| 18 | 3% | 25 mM | 4.63 | 4.8 | 6 | 4.7 | 4.8 | 5.1 | NT | 5.6 |
| | Sucrose | Leu, 50 | | | | | | | | |
| | | mM Gly | | | | | | | | |

NT = “Not tested”

### Example 7. Evaluation of Additional Glutamate Formulations of FXX01102 at −20° C.

An experiment was performed to evaluate the stability of FXX01102 when stored at 20° C. All formulations shown in Table 4 below contained 5 mM glutamate as the buffering agent. In addition, all formulations contained a sugar component, either 0, 2, 3, or 8% w/v sucrose. Based on previous data, we further investigated variations in Leu concentration was well as pH on the stability of FXX01102. It was performed in the same manner as was described in Example 6. A formulation with improved stability should show less aggregate formation as measured by size exclusion ultra pressure liquid chromatography (SE-UPLC). The data was analyzed for differences in % HMW between the initial and 6 week timepoints as well as a 3×F/T stress test. The data showed that lower pH values and higher concentrations of leucine inhibited aggregate formation over time as well as during multiple freeze thaw cycles. Inclusion of sucrose in the formulation had a positive effect with 10 mM glutamate as buffering agent and 0.02% Polysorbate. In addition, all formulations contained a sugar component, either 3 or 8% w/v sucrose, 3% trehalose or 3% sorbitol. Based on previous data, we also investigated the addition of Leu, Met or Gly amino acids, whereas the absence of sucrose resulted in a higher % HMW than other formulations (Formulation #6, Table 4). Similar to the previous experiment, higher levels of sucrose had a nominal impact on the storage stability data at 6 weeks, but had a greater impact on inhibiting aggregate formation during the 3×F/T,

TABLE 4
Buffers evaluated for impact on % HMW after storage at −20 ° C.

| | | [Protein] | SE-UPLC | | | |
|---|---|---|---|---|---|---|
| Time | | (mg/ml) | % HMW | | | |
| Point | Formulation | Initial | Initial | 3 Week | 3X FT | 6 Week |
| 1 | 3% Suc, 0.02% PS80, pH = 4.65 | 8.65 | 0.87 | 1.83 | 2.84 | 2.72 |
| 2 | 3% Suc, 0.02% PS80, pH = 4.66 | 8.62 | 1.05 | 1.91 | 3.07 | 2.53 |
| 3 | 3% Suc, 0.02% PS80, pH = 4.86 | 8.67 | 2.08 | 2.95 | 5.13 | 3.55 |
| 4 | 3% Suc, 25 mM Leu, 0.02% PS80, pH = 4.69 | 8.56 | 1.2 | 1.33 | 2.05 | 1.38 |
| 5 | 3% Suc, 37.5 mM Leu, 0.02% PS80, pH = | 8.32 | 1.19 | 1.31 | 1.87 | 1.38 |
| | 4.74 | | | | | |
| 6 | 0% Suc, 0.02% PS80, pH = 4.62 | 8.57 | 1.05 | 2.75 | 8.18 | 2.83 |
| 7 | 3% Suc, 0.05% PS80, pH = 4.66 | 8.8 | 1.07 | 2.34 | 3.44 | 3.09 |
| 8 | 8% Suc, 0.02% PS80, pH = 4.63 | 8.61 | 1.05 | 1.65 | 2.08 | 1.98 |
| 9 | 3% Suc, 25 mM Leu, 0.02% PS80, pH = 4.48 | 9.61 | 0.71 | 0.78 | 0.91 | 0.82 |
| 10 | 3% Suc, 25 mM Leu, 0.02% PS80, pH = 4.73 | 9.6 | 1.21 | 1.36 | 1.63 | 1.34 |
| 11 | 3% Suc, 25 mM Leu, 0.02% PS80, pH = 4.81 | 9.72 | 2.33 | 2.48 | 3.62 | 2.68 |
| 12 | 3% Suc, 25 mM Leu, 0.02% PS80, pH = 4.70 | 9.63 | 1.03 | 1.14 | 1.41 | 1.15 |
| 13 | 3% Suc, 25 mM Leu, 0.02% PS80, pH = 4.57 | 9.69 | 1.61 | 1.71 | 2.28 | 1.72 |
| 14 | 3% Suc, 37.5 mM Leu, 0.02% PS80, pH = | 9.62 | 1.36 | 1.37 | 1.85 | 1.51 |
| | 4.57 | | | | | |
| 15 | 3% Suc, 50 mM Leu, 0.02% PS80, pH = 4.58 | 9.48 | 1.42 | 1.44 | 2.02 | 1.47 |
| 16 | 3% Suc, 25 mM Leu, 0.05% PS80, pH = 4.61 | 9.65 | 1.21 | 1.28 | 1.66 | 1.39 |
| 17 | 2% Suc, 25 mM Leu, 0.02% PS80, pH = 4.55 | 9.71 | 1.22 | 1.29 | 1.79 | 1.29 |
| 18 | 5% Suc, 25 mM Leu, 0.02% PS80, pH = 4.55 | 9.63 | 1.18 | 1.28 | 1.48 | 1.45 |
| 19 | 3% Suc, 10 mM Leu, 0.02% PS80, pH = 4.58 | 8.51 | 1.06 | 1.1 | 1.37 | 1.18 |
| 20 | 3% Suc, 37.5 mM Leu, 0.02% PS80, pH = | 9.74 | 1.68 | 1.09 | 1.05 | 1.09 |
| | 4.53 | | | | | |
| 21 | 3% Suc, 37.5 mM Leu, 0.02% PS80, pH = | 9.87 | 2.11 | 1.6 | 1.95 | 1.61 |
| | 4.61 | | | | | |
| 22 | 3% Suc, 37.5 mM Leu, 0.02% PS80, pH = | 10.03 | 2.63 | 2.53 | 3.39 | 2.67 |
| | 4.85 | | | | | |
| 23 | 3% Suc, 50 mM Leu, 0.02% PS80, pH = 4.57 | 9.84 | 1.75 | 1.19 | 1.38 | 1.19 |
| 24 | 3% Suc, 50 mM Leu, 0.02% PS80, pH = 4.48 | 9.79 | 2.16 | 1.7 | 2.02 | 1.72 |

The invention is not to be limited in scope by the specific aspects described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Other aspects are within the following claims.

SEQUENCES
SEQ ID NO: 1
GYTFTSYW
SEQ ID NO: 2
IYPGSSTT
SEQ ID NO: 3
ASFSDGYYAYAMDY
SEQ ID NO: 4
QDISNY
SEQ ID NO: 5
YTS
SEQ ID NO: 6
QQGYTLPYT
SEQ ID NO: 7
GFTLSYYG
SEQ ID NO: 8
ISHDGSDK
SEQ ID NO: 9
SNDQFDP
SEQ ID NO: 10
NIGSKS
SEQ ID NO: 11
DDS
SEQ ID NO: 12
QVWDSSSDHVV
SEQ ID NO: 13
EVQLVQSGAEVKKPGASVKVSCKASGYTFTSYWMNWVRQAPGQGLEWMGN
IYPSGGSTNYAQKFQGRVTMTVDTSTSTVYMELSSLRSEDTAVYYCASFS
DGYYAYAMDYWGQGTLVTVSSGGGGSGGGGSGGGGSGGGGSEIVMTQSPA
TLSLSPGERATLSCRASQSVSSYLNWYQQKPGQAPRLLIYYASRRHTGIP
ARFSGSGSGTDFTLTISSLQPEDFAVYYCQQGYNLPYTFGQGTKVEIKEP
KSSDKTHTCPPCPAPPAAAPSVFLFPPKPKDTLMISRTPEVTCVVVDVSH
EDPEVKFNWYVDGVEVHNAKTKPREEQYNSTYRVVSVLTVLHQDWLNGKE
YKCAVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVFSCSVMHEALHNHYTQKSLSLSPGGGGSPSSYVLTQPPSVSVAPGK
TARITCGGNNIGSKSVNWFQQKPGQAPVLVVYDDSGRPSGIPERFSGSTS
GNTATLTISRVEAGDEADYYCQVWDSSSDHVVFGGGTKLTVLGGGGSGGG
GSGGGGSGGGGSQVQLVESGGGVVQPGRSLRLSCAASGFTLSYYGMHWVR
QAPGKGLEWVAAISHDGSDKYYADSVKGRFTISRDNSKNRLYLQMNSLRA
EDTAVYYCSNDQFDPWGQGTLVTVSSR
SEQ ID NO: 14
EVQLVQSGAEVKKPGASVKVSCKASGYTFTSYWMNWVRQAPGQGLEWMGN
IYPSGGSTNYAQKFQGRVTMTVDTSTSTVYMELSSLRSEDTAVYYCASFS
DGYYAYAMDYWGQGTLVTVSS
SEQ ID NO: 15
EIVMTQSPATLSLSPGERATLSCRASQSVSSYLNWYQQKPGQAPRLLIYY
ASRRHTGIPARFSGSGSGTDFTLTISSLQPEDFAVYYCQQGYNLPYTFGQ
GTKVEIK
SEQ ID NO: 16
EVQLVQSGAEVKKPGASVKVSCKASGYTFTSYWMNWVRQAPGQGLEWMGN
IYPSGGSTNYAQKFQGRVTMTVDTSTSTVYMELSSLRSEDTAVYYCASFS
DGYYAYAMDYWGQGTLVTVSSGGGGSGGGGSGGGGSGGGGSEIVMTQSPA
TLSLSPGERATLSCRASQSVSSYLNWYQQKPGQAPRLLIYYASRRHTGIP
ARFSGSGSGTDFTLTISSLQPEDFAVYYCQQGYNLPYTFGQGTKVEIK
SEQ ID NO: 17
QVQLVESGGGVVQPGRSLRLSCAASGFTLSYYGMHWVRQAPGKGLEWVAA
ISHDGSDKYYADSVKGRFTISRDNSKNRLYLQMNSLRAEDTAVYYCSNDQ
FDPWGQGTLVTVSS
SEQ ID NO: 18
SYVLTQPPSVSVAPGKTARITCGGNNIGSKSVNWFQQKPGQAPVLVVYDD
SGRPSGIPERFSGSTSGNTATLTISRVEAGDEADYYCQVWDSSSDHVVFG
GGTKLTVL
SEQ ID NO: 19
SYVLTQPPSVSVAPGKTARITCGGNNIGSKSVNWFQQKPGQAPVLVVYDD
SGRPSGIPERFSGSTSGNTATLTISRVEAGDEADYYCQVWDSSSDHVVFG
GGTKLTVLGGGGSGGGGSGGGGSGGGGSQVQLVESGGGVVQPGRSLRLSC
AASGFTLSYYGMHWVRQAPGKGLEWVAAISHDGSDKYYADSVKGRFTISR
DNSKNRLYLQMNSLRAEDTAVYYCSNDQFDPWGQGTLVTVSSR
SEQ ID NO: 20
GGGGSGGGGSGGGGS
SEQ ID NO: 21
GGGGS

1. A pharmaceutical composition comprising:
(a) a bispecific antibody or antigen-binding fragment thereof that specifically binds 4-1BB and OX40;
(b) about 5 mM to about 15 mM of a stability promoting buffer;
(c) about 25 mM to about 50 mM of a stabilizing amino acid;
(d) about 2% to about 8% w/v of a sugar; and
(e) about 0.01% to about 0.03% of a surfactant.

2. The pharmaceutical composition of claim 1, wherein the stability promoting buffer is selected from the group consisting of succinate, histidine, citrate, and glutamate.

3. The pharmaceutical composition of claim 1 or 2, wherein the stability promoting buffer is present in the composition in a concentration of about 5 mM, about 6 mM, about 7 mM, about 8 mM, about 9 mM, about 10 mM, about 11 mM, about 12 mM, about 13 mM, about 14 mM, or about 15 mM.

4. The pharmaceutical composition of claim 3, wherein the stability promoting buffer is glutamate.

5. The pharmaceutical composition of any one of claims 2-4, wherein the glutamate is present in the composition in a concentration of about 5 mM.

6. The pharmaceutical composition of any of claims 1-5, wherein the stabilizing amino acid is selected from the group consisting of arginine, methionine, leucine, and glycine.

7. The pharmaceutical composition of claim 6, wherein the stabilizing amino acid is present in the composition in a concentration of about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, or about 50 mM.

8. The pharmaceutical composition of claim 6 or 7, wherein the stabilizing amino acid is leucine.

9. The pharmaceutical composition of any one of claims 6-8, wherein the leucine is present in the composition in a concentration of about 40 mM.

10. The pharmaceutical composition of any one of claims 1-9, wherein the sugar is selected from the group consisting of: sucrose, trehalose, and sorbitol.

11. The pharmaceutical composition of claim 10, wherein the sugar is present in the composition in a concentration from about 2% w/v to about 8% w/v.

12. The pharmaceutical composition of any one of claims 10 or 11, wherein the sugar is present in the composition in a concentration of about 2% w/v, about 3% w/v, about 4% w/v, about 5% w/v, about 6% w/v, about 7% w/v, or about 8% w/v.

13. The pharmaceutical composition of any one of claims 10-12, wherein the sugar is sucrose.

14. The pharmaceutical composition of any one of claims 11-13, wherein the sucrose is present in the composition in a concentration of about 3% w/v.

15. The pharmaceutical composition of any one of claims 1-14, further comprising a surfactant.

16. The pharmaceutical composition of claim 15, wherein the surfactant is Polysorbate-80.

17. The pharmaceutical composition of claim 16, wherein the Polysorbate-80 is present in the composition in a concentration of from about 0.01% w/v to about 0.03% w/v.

18. The pharmaceutical composition of claim 6 or 17, wherein the Polysorbate-80 is present in the composition in a concentration of about 3% w/v.

19. The pharmaceutical composition of any one of claims 1-18, wherein the pH of the composition is in a range from about 4.3 to about 4.7.

20. The pharmaceutical composition of any one of claims 1-19, wherein the pH of the composition is about 4.5.

21. The pharmaceutical composition of any one of claims 1-20, wherein the bispecific antibody or antigen-binding fragment thereof is present in the composition in a concentration of about 10 mg/mL to about 50 mg/mL.

22. The pharmaceutical composition of any one of claims 1-21, wherein the pharmaceutical composition does not comprise sodium chloride (NaCl).

23. The pharmaceutical composition of any one of claims 1-22, wherein the 4 bispecific antibody or antigen-binding fragment thereof has a purity of about >90%.

24. The pharmaceutical composition of any one of claims 1-23, comprising:
(a) the 4-1BB×OX40 bispecific antibody or antigen-binding fragment thereof in a concentration of about 10 mg/mL;
(b) about 10 mM glutamate;
(c) about 40 mM leucine;
(d) about 3% w/v sucrose; and
(e) about 0.02% polysorbate-80
wherein the pH of the composition is from about 4.3 to about 4.7.

25. The pharmaceutical composition of any one of claims 1-24, comprising:
(a) the 4-1BB×OX40 bispecific antibody or antigen-binding fragment thereof in a concentration of 10 mg/mL;
(b) 10 mM glutamate;
(c) 40 mM leucine;
(d) 3% w/v sucrose; and
(e) 0.02% polysorbate-80;
wherein the pH of the composition is 4.5.

26. The pharmaceutical composition of any one of claims 1-25, wherein the bispecific antibody or antigen-binding fragment thereof comprises a single chain Fv (scFv).

27. The pharmaceutical composition of any one of claims 1-26, wherein the bispecific antibody or antigen-binding fragment thereof comprises a Fab, Fab′, F(ab′)2, scFv, disulfide linked Fv, or scFv-Fc.

28. The pharmaceutical composition of any one of claims 1-27, wherein the bispecific antibody or antigen-binding fragment thereof comprises a human 4-1BB antigen-binding domain and a human OX40 antigen-binding domain, wherein:
(a) the 4-1BB antigen-binding domain comprises a (i) a VH-CDR 1 comprising the amino acid sequence of SEQ ID NO:1; (ii) a VH-CDR2 comprising the amino acid sequence of SEQ ID NO:2; (iii) a VH-CDR3 comprising the amino acid sequence of SEQ ID NO:3; (iv) a light chain variable domain (VL)-CDR1 comprising the amino acid sequence of SEQ ID NO:4; (v) a VL-CDR2 comprising the amino acid sequence of SEQ ID NO:5; and (vi) a VL-CDR3 comprising the amino acid sequence of SEQ ID NO:6; and
(b) the OX40 antigen-binding domain comprises a (i) a VH-CDR1 comprising the amino acid sequence of SEQ ID NO:7; (ii) a VH-CDR2 comprising the amino acid sequence of SEQ ID NO:8; (iii) a VH-CDR3 comprising the amino acid sequence of SEQ ID NO:9; (iv) a VL-CDR1 comprising the amino acid sequence of SEQ ID NO:10; (v) a VL-CDR2 comprising the amino acid sequence of SEQ ID NO:11; and (vi) a VL-CDR3 comprising the amino acid sequence of SEQ ID NO:12.

29. The pharmaceutical composition of any one of claims 1-28, wherein the bispecific antibody or antigen-binding fragment thereof comprises a human 4-1BB antigen-binding domain and a human OX40 antigen-binding domain, wherein:
(a) the 4-1BB antigen-binding domain comprises a heavy chain variable region comprising the amino acid sequence of SEQ ID NO: 14 and a light chain variable region comprising the amino acid sequence of SEQ ID NO: 15; and
(b) the OX40 antigen-binding domain comprises a heavy chain variable region comprising the amino acid sequence of SEQ ID NO: 17 and a light chain variable region comprising the amino acid sequence of SEQ ID NO: 18.

30. The pharmaceutical composition of any one of claims 1-29, wherein the 4-1BB×OX40 bispecific antibody or antigen-binding fragment thereof comprises a human 4-1BB antigen-binding domain and a human OX40 antigen-binding domain, wherein:
(a) the 4-1BB antigen-binding domain comprises a scFv comprising the amino acid sequence of SEQ ID NO: 16; and
(b) the OX40 antigen-binding domain comprises a scFv comprising the amino acid sequence of SEQ ID NO: 19.

31. The pharmaceutical composition of any one of claims 1-30, wherein the bispecific antibody or antigen-binding fragment thereof comprises the amino acid sequence of SEQ ID NO: 13.

32. A pharmaceutical composition, comprising:
(a) 10 mg/mL of a 4-1BB×OX40 bispecific antibody or antigen-binding fragment thereof, wherein the antibody or antigen-binding fragment thereof comprises:
(i) a 4-1BB antigen-binding domain comprising a VH-CDR 1 comprising the amino acid sequence of SEQ ID NO:1; a VH-CDR2 comprising the amino acid sequence of SEQ ID NO:2; a VH-CDR3 comprising the amino acid sequence of SEQ ID NO:3; a light chain variable domain (VL)-CDR1 comprising the amino acid sequence of SEQ ID NO:4; a VL-CDR2 comprising the amino acid sequence of SEQ ID NO:5; and a VL-CDR3 comprising the amino acid sequence of SEQ ID NO:6; and
an OX40 antigen-binding domain comprising a VH-CDR1 comprising the amino acid sequence of SEQ ID NO:7; a VH-CDR2 comprising the amino acid sequence of SEQ ID NO:8; a VH-CDR3 comprising the amino acid sequence of SEQ ID NO:9; a VL-CDR1 comprising the amino acid sequence of SEQ ID NO:10; a VL-CDR2 comprising the amino acid sequence of SEQ ID NO: 11; and a VL-CDR3 comprising the amino acid sequence of SEQ ID NO:12;
(ii) a 4-1BB antigen-binding domain comprising a heavy chain variable region comprising the amino acid sequence of SEQ ID NO: 14 and a light chain variable region comprising the amino acid sequence of SEQ ID NO: 15; and
a OX40 antigen-binding domain comprising a heavy chain variable region comprising the amino acid sequence of SEQ ID NO: 17 and a light chain variable region comprising the amino acid sequence of SEQ ID NO: 18;
(iii) a 4-1BB antigen-binding domain comprising a scFv comprising the amino acid sequence of SEQ ID NO: 16; and an OX40 antigen-binding domain comprising a scFv comprising the amino acid sequence of SEQ ID NO: 19; or
(iv) the amino acid sequence of SEQ ID NO: 13;
(b) about 5 mM to about 15 mM glutamate;
(c) about 25 mM to about 50 mM leucine;
(d) about 2% to about 8% w/v sucrose; and
(e) about 0.01% to about 0.03% polysorbate-80;
wherein the pH of the composition is from about 4.3 to about 4.7.

33. A pharmaceutical composition, comprising:
(a) 10 mg/mL of a 4-1BB×OX40 bispecific antibody or antigen-binding fragment thereof; wherein the antibody or antigen-binding fragment thereof comprises:
(i) a 4-1BB antigen-binding domain comprising a VH-CDR 1 comprising the amino acid sequence of SEQ ID NO:1; a VH-CDR2 comprising the amino acid sequence of SEQ ID NO:2; a VH-CDR3 comprising the amino acid sequence of SEQ ID NO:3; a light chain variable domain (VL)-CDR1 comprising the amino acid sequence of SEQ ID NO:4; a VL-CDR2 comprising the amino acid sequence of SEQ ID NO:5; and a VL-CDR3 comprising the amino acid sequence of SEQ ID NO:7; and
(ii) an OX40 antigen-binding domain comprising a VH-CDR1 comprising the amino acid sequence of SEQ ID NO:7; a VH-CDR2 comprising the amino acid sequence of SEQ ID NO:8; a VH-CDR3 comprising the amino acid sequence of SEQ ID NO:9; a VL-CDR1 comprising the amino acid sequence of SEQ ID NO:10; a VL-CDR2 comprising the amino acid sequence of SEQ ID NO: 11; and a VL-CDR3 comprising the amino acid sequence of SEQ ID NO:12;
(b) 10 mM glutamate;
(c) 40 mM leucine;
(d) 3% w/v sucrose; and
(e) 0.02% polysorbate-80;
wherein the pH of the composition is 4.5.

34. The pharmaceutical composition of claim 33, wherein
(a) the 4-1BB antigen-binding domain comprises a heavy chain variable region comprising the amino acid sequence of SEQ ID NO: 14 and a light chain variable region comprising the amino acid sequence of SEQ ID NO: 15; and
(b) the OX40 antigen-binding domain comprises a heavy chain variable region comprising the amino acid sequence of SEQ ID NO: 17 and a light chain variable region comprising the amino acid sequence of SEQ ID NO: 18.

35. The pharmaceutical composition of any one of claims 1-34, wherein the pharmaceutical formulation is a liquid.

36. The pharmaceutical composition of any one of claims 28-30 and 32-35, wherein the human 4-1BB binding domain and the human OX40 binding domain are on the same polypeptide.

37. The pharmaceutical composition of claim 36, wherein the human 4-1BB binding domain is N-terminal to the human OX40 binding domain.

38. The pharmaceutical composition of claim 36, wherein the human 4-1BB binding domain is C-terminal to the human OX40 binding domain.

39. The pharmaceutical composition of any one of claims 1-30 and 32-38, wherein the antibody or antigen-binding fragment thereof comprises an immunoglobulin constant region.

40. The pharmaceutical composition claim 39, wherein the immunoglobulin constant region comprises immunoglobulin CH2 and CH3 domains of IgG1.

41. The pharmaceutical composition of any one of claims 1-40, wherein the antibody does not contain a CH1 domain.

42. The pharmaceutical composition of any one of claims 39-41, wherein the immunoglobulin constant region comprises a human IgG1 CH2 domain comprising the substitutions E233P, L234A, L235A, G237A, and K322A and a deletion of G236 according to the EU numbering system.

43. The pharmaceutical composition of any one of claims 28-30 and 32-42, wherein the antibody or antigen-binding fragment thereof comprises a linker between an immunoglobulin constant region and the human 4-1BB binding domain and/or between an immunoglobulin constant region and the human OX40 binding domain.

44. The pharmaceutical composition of claim 43, wherein the linker between the immunoglobulin constant region and the human 4-1BB binding domain and/or between the immunoglobulin constant region and the human OX40 binding domain comprises 10-30 amino acids, 15-30 amino acids, or 20-30 amino acids.

45. The pharmaceutical composition of claim 44, wherein the linker between the immunoglobulin constant region and the human 4-1BB binding domain or between the immunoglobulin constant region and the human OX40 binding domain comprises the amino acid sequence (Gly4Ser)n, wherein n=1-5 (SEQ ID NO:21), optionally wherein n=1.

46. The pharmaceutical composition of any one of claims 28-45, wherein the antibody comprises a dimer of two polypeptides, each polypeptide comprising in order from amino-terminus to carboxyl-terminus, a first scFv, a hinge region, an immunoglobulin constant region, and a second scFv, wherein (a) the first scFv comprises a human 4-1BB antigen-binding domain, and the second scFv comprises a human OX40 antigen-binding domain or (b) the first scFv comprises a human OX40 antigen-binding domain and the second scFv comprises a human 4-1BB antigen-binding domain.

47. The pharmaceutical composition of claim 46, wherein the dimer is a homodimer.

48. The pharmaceutical composition of any one of claims 1-47, wherein the pharmaceutical formulation can be stored at 20° C.

49. The pharmaceutical composition of any one of claims 1-48, having no significant change in pH after storage at 20° C., after about 3 weeks, about 6 weeks, about 3 months, about 6 months, and about 7 months.

50. The pharmaceutical composition of any one of claims 1-49, having no significant change in the percent High Molecular Weight content (% HMW) after storage at 20° C., after about 3 weeks, about 6 weeks, about 3 months, about 6 months, or about 7 months.

51. The pharmaceutical composition of any one of claims 1-50, wherein the change in the % HMW after storage at −20° C., after about 3 weeks, about 6 weeks, about 3 months, about 6 months, or about 7 months is less than 5%.

52. The pharmaceutical composition of any one of claims 1-51, having no significant change in the % HMW after one, two, or three freeze/thaw cycles.

53. The pharmaceutical composition of any one of claims 1-52, wherein the change in the % HMW after one, two, or three freeze/thaw cycles is less than 5%.

54. The pharmaceutical composition of any one of claims 50-53, wherein the % HMW is measured by size exclusion ultra pressure liquid chromatography (SE-UPLC).

55. The pharmaceutical composition of any one of claims 1-54, having no significant change in aggregate formation after storage at 20° C., after about 3 weeks, about 6 weeks, about 3 months, about 6 months, or about 7 months, as measured by size exclusion ultra pressure liquid chromatography (SE-UPLC).

56. A method of treating cancer in a subject, the method comprising administering to the subject an effective amount of the pharmaceutical composition of any one of claims 1-55.

57. The method of claim 56, wherein the cancer is a solid tumor cancer.

58. The method of claim 56 or 57, wherein the cancer is a sarcoma, a carcinoma, or a lymphoma.

59. The method of any one of claims 56-58, wherein the cancer is selected from the group consisting of a melanoma, kidney cancer, pancreatic cancer, lung cancer, stomach cancer, colon/intestinal cancer, prostate cancer, ovarian cancer, breast cancer, liver cancer, brain cancer, or a hematological cancer.

60. The method of any one of claims 56-59, wherein the subject is a human.

61. The method of any one of claims 56-60, wherein the subject expresses 4-1BB and OX40 on tumor infiltrating lymphocytes.

62. The pharmaceutical composition of any one of claims 1-61 for use in the treatment of cancer.

63. The pharmaceutical composition of any one of claims 1-62 for use in the treatment of a solid tumor cancer.

64. The antibody pharmaceutical composition for the use of claim 63, wherein the solid tumor cancer is a sarcoma, carcinoma, or lymphoma.

65. The pharmaceutical composition for the use of claim 64, wherein the cancer is selected from the group consisting of a melanoma, kidney cancer, pancreatic cancer, lung cancer, colon/intestinal cancer, stomach cancer, prostate cancer, ovarian cancer, breast cancer, liver cancer, brain cancer, or a hematological cancer.
